SEQUENCE LISTING (16 sequences)

SEQ ID NO: 1 - protein, 6 aa, Mus sp.
VAYEEL

SEQ ID NO: 2 - protein, 6 aa, Unknown
  Description of Unknown Human or mouse ITIM-like motif
TEYATI

SEQ ID NO: 3 - protein, 6 aa, Artificial Sequence
  Description of Artificial Sequence Synthetic 6xHis tag
HHHHHH

SEQ ID NO: 4 - protein, 18 aa, Artificial Sequence
  Description of Artificial Sequence Synthetic peptide
SASSKHTNLYWSRHMYWY

SEQ ID NO: 5 - protein, 7 aa, Artificial Sequence
  Description of Artificial Sequence Synthetic peptide
LTSNRAT

SEQ ID NO: 6 - protein, 7 aa, Artificial Sequence
  Description of Artificial Sequence Synthetic peptide
QQWSSNP

SEQ ID NO: 7 - protein, 10 aa, Artificial Sequence
  Description of Artificial Sequence Synthetic peptide
GFTFSDYGMH

SEQ ID NO: 8 - protein, 17 aa, Artificial Sequence
  Description of Artificial Sequence Synthetic peptide
YISSGSYTIYSADSVKG

SEQ ID NO: 9 - protein, 13 aa, Artificial Sequence
  Description of Artificial Sequence Synthetic peptide
RAPNSFYEYYFDY

SEQ ID NO: 10 - protein, 195 aa, Artificial Sequence
  Description of Artificial Sequence Synthetic polypeptide
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHK

SEQ ID NO: 11 - protein, 452 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 10 aa, Artificial Sequence
  Description of Artificial Sequence Synthetic peptide
SASSSVSYMY

SEQ ID NO: 13 - protein, 9 aa, Artificial Sequence
  Description of Artificial Sequence Synthetic peptide
QQWSSNPFT

SEQ ID NO: 14 - protein, 12 aa, Artificial Sequence
  Description of Artificial Sequence Synthetic peptide
RGYGSFYEYYFD

SEQ ID NO: 15 - protein, 108 aa, Artificial Sequence
  Description of Artificial Sequence Synthetic polypeptide
QIVLTQSPATLSLSPGERATLSCSASSSVSYMYWYQQKPGQAPRLLIYLTSNRATGIPARFSGSGSGTDYSSTLTISSLEPEDFAVYYCQQWSSNPFTFGQGTKLEIK

SEQ ID NO: 16 - protein, 122 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNTLYLQMSSLRAEDTAVYYCARRGYGSFYEYYFDYWGQGTTVTVSS

(19) United States
(12) Patent Application Publication
(10) Pub. No.: US 2020/0199567 A1
(43) Pub. Date: Jun. 25, 2020

(54) METHODS FOR SELECTION AND EXPANSION OF T CELLS EXPRESSING PD-1

(71) Applicant: MEDIMMUNE, LLC, Gaithersburg, MD (US)

(72) Inventors: SHINO HANABUCHI, GAITHERSBURG, MD (US); JOHN MUMM, GAITHERSBURG, MD (US); DANIEL J. FREEMAN, GAITHERSBURG, MD (US); JINLIN JIANG, GAITHERSBURG, MD (US); SOMEET NARANG, GAITHERSBURG, MD (US); RONALD HERBST, GAITHERSBURG, MD (US); DANIELLE TOWNSLEY, GAITHERSBURG, MD (US); GIANLUCA CARLESSO, GAITHERSBURG, MD (US); TAEIL KIM, GAITHERSBURG, MD (US)

(21) Appl. No.: 16/718,920

(22) Filed: Dec. 18, 2019

Related U.S. Application Data
(60) Provisional application No. 62/782,537, filed on Dec. 20, 2018.

(51) Int. Cl. C12N 13/00; C12N 5/0783; G01N 33/543; A61K 35/17; A61K 39/395

(52) U.S. Cl. CPC C12N 13/00; C12N 5/0636; A61K 39/3955; A61K 35/17; G01N 33/54326

(57) ABSTRACT

The disclosure provides methods for the selection and isolation of T cells expressing programmed cell death 1 (PD-1) and for selecting a PD-1 expression level of the isolated PD-1 expressing T cells. The disclosure also provides methods of large scale expansion of selected and isolated PD-1 expressing T cells, as well as methods for treating a subject comprising administering selected and isolated PD-1 expressing T cells to the subject.

### BACKGROUND

It has recently been reported that PD-1 expression on both tumor infiltrating and peripheral blood T cells in cancer patients is enriched for tumor antigen reactivity. Programmed Death-1 (PD-1) was originally identified as a marker of previously activated T cells that are enroute to apoptosis. Recently, this view of PD-1 has begun to shift to a view where PD-1 expression is now thought to represent a marker of T cell activation, and only demarcates T cell exhaustion or a harbinger of apoptosis when expressed in context of TIM3 or LAG3. In particular, recent data suggests that PD-1+ cells in tumor infiltrating lymphocytes (TIL) and peripheral blood represent the same neoatigen (NeoAg) reactive T cell populations (Gros et al., 2014, J. Clin. Invest. 124(5): 2246-59). These data suggest PD-1 expression may correlate with T cells that have been exposed to and have responded to tumor antigens.

However, it is unknown how the levels of PD-1 expression correlate with either NeoAg reactivity or the time point at which T cells may have encountered antigen. While fluorescent activated cell sorting (FACS) can be utilized to isolate specific cells employing antibodies to cell surface proteins, the disclosure herein describes that isolating PD-1+ T cells from peripheral blood via FACS by targeting PD-1 expressing cells yielded cells that were deficient in their capacity to expand ex vivo.

The isolation, ex vivo expansion, and autologous reinfusion of TIL from melanoma patients has been explored in multiple clinical trials and has demonstrated a 50% overall response rate, suggesting that the ex vivo expansion of T cells is a viable therapeutic intervention (Dudley et al., 2010, Clin. Can. Res. 16(24): 6122-31). Several methodologies have been developed to expand isolated T cells ex vivo, which primarily utilize irradiated feeder cells that function to provide immobilized co-stimulatory molecules. These include the use of high dose Interleukin-2 (IL-2), which is often combined with high dose Interleukin-15 (IL-15) and/or Interleukin-17 (IL-7).

While this rapid expansion protocol, (Dudley et al., 2003, J. Immunother. 26(4): 332-42) is currently used in the field for expansion of chimeric antigen receptor T cells, the process is not amenable to treatment of more than a few thousand patients. In particular, the use of irradiated feeder cells precludes the use of this protocol in most hospitals, which are not commonly equipped with cell culture or irradiation facilities. In addition, contamination of cellular debris present at the end of the culture conditions confounds drug product release. High concentrations of IL-2 drive uncontrollable T cell proliferation, decoupling the T cell receptor (TCR) repertoire of the starting culture from the TCR repertoire post expansion. This similarly confounds drug product release.

Lastly, the supply of human serum is variable and represents an additional uncontrollable manufacturing element. It is also known that the existing lot-to-lot variability of serum can potentially expose recipient patients to accidental infectious pathogens. The degree to which these changes alter T cell growth characteristics is unknown. It is also known in the field that a high degree of variability exists with regard to T cell growth characteristics, where in any given rapid expansion culture, the CD8 or CD4 T cells can overgrow the culture, or the relative ratio of CD4 to CD8 that was in the initial culture is maintained through the expansion. Collectively, these manufacturing risks indicate a pressing need for a simplified ex vivo rapid T cell expansion protocol that is scalable, controllable, and reproducible, preferably maintaining the CD4/CD8 ratio throughout the expansion.

Therefore as described above, T cells that express PD-1 hold potential for therapeutic use due to their tumor antigen specificity. By isolating these cells from cancer patients based on their PD-1 expression levels, the tumor antigen reactive cells can be enriched, expanded, and re-invigorated ex vivo to provide a superior activated cellular product when compared to both the TIL (not sufficiently tumor antigen reactive enriched) or CART (monoAg targeted, potentially overactived) cell therapy products that are currently being explored. Accordingly, there is a need for methods of selecting and expanding PD-1 expressing T cells.

### SUMMARY

The disclosure provides a method of isolating T cells expressing programmed cell death 1 (PD-1) from a cell population, comprising: (a) contacting the cell population with an amount of an anti-PD-1 antibody to produce an antibody-cell mixture, wherein the anti-PD-1 antibody comprises a capture moiety, and wherein the capture moiety is connected to the anti-PD-1 antibody via a linker; (b) contacting the antibody-cell mixture with an amount of magnetic beads, wherein the magnetic beads are capable of specifically binding the capture moiety on the anti-PD-1 antibody to produce a bead mixture; (c) passing the bead mixture through a magnetic field to isolate the magnetic beads and PD-1 expressing T cells bound thereto from the bead mixture; and (d) eluting the PD-1 expressing T cells from the magnetic field to isolate T cells expressing PD-1.

The disclosure also provides a method of treating a subject comprising administering to the subject a therapeutically effective amount of T cells expressing PD-1; wherein the T cells expressing PD-1 are isolated by: (a) contacting the cell population with an amount of an anti-PD-1 antibody to produce an antibody-cell mixture, wherein the anti-PD-1 antibody comprises a capture moiety, and wherein the capture moiety is connected to the anti-PD-1 antibody via a linker; (b) contacting the antibody-cell mixture with an amount of magnetic beads, wherein the magnetic beads are capable of specifically binding the capture moiety on the anti-PD-1 antibody to produce a bead mixture; (c) passing the bead mixture through a magnetic field to isolate the magnetic beads and PD-1 expressing T cells bound thereto from the bead mixture; and (d) eluting the PD-1 expressing T cells from the magnetic field to isolate T cells expressing PD-1; wherein a PD-1 expression level of the T cells expressing PD-1 isolated in step (d) is selected by adjusting one or more of: (i) the concentration of the anti-PD-1 antibody in the antibody-cell mixture; (ii) the length of the linker; (iii) the stoichiometric ratio of capture moiety to anti-PD-1 antibody (CAR); (iv) the ratio of anti-PD-1 antibody comprising a capture moiety to unmodified anti-PD-1 antibody; (v) the temperature at which either step (a) and/or step (b) is carried out; (vi) the concentration of T cells in the cell population; (vii) the concentration of magnetic beads in the bead mixture; (viii) the flow rate at which the bead mixture is passed through the magnetic field; (ix) the length of time between the production of the antibody-cell mixture and step (b); (x) the length of time between production of the bead mixture and step (c); or (xi) the force applied by the magnetic field; and wherein the population of T cells expressing PD-1 is subjected to ex vivo expansion by: (a) priming a sample of T cells expressing PD-1 isolated according to the methods disclosed herein, wherein the step of priming comprises: (i) coating a culture plate on day −1 with priming factors; (ii) seeding the population of T cells expressing PD-1 on day 0, wherein the step of seeding comprises: (1) adding a base media to the coated culture plate; and/or (2) adding an amount of isolated and/or enriched T cells to the base media to produce a seeding mixture in the coated culture plate; (b) harvesting the primed T cells expressing PD-1; (c) placing the harvested T cells expressing PD-1 in a seeding mixture and placing the seeding mixture into a non-treated culture plate; (d) culturing the T cells expressing PD-1 in the seeding mixture; (e) harvesting the T cells expressing PD-1 from the cultured seeding mixture; (f) repeating steps (b)-(e) until a target number of expanded T cells is obtained.

In some embodiments of any of the methods of expansion and/or treatment disclosed herein, the priming factors used in T cell expansion include an ICOS agonist. In some embodiments, the ICOS agonist is an anti-ICOS antibody. In some embodiments, T cells are primed for 4 days. In some embodiments, culturing of T cells after priming is conducted in the presence of IL-2.

In another aspect, the disclosure provides methods of treating cancer in a subject, the methods comprising: (a) administering an anti-CTLA-4 antibody to the subject in an amount effective to mobilize PD-1⁺ tumor-infiltrating lymphocytes (TIL) into the peripheral blood; (b) harvesting PD-1⁺ T cells (e.g., TIL) from the peripheral blood of the subject; (c) expanding the harvested PD-1⁺ T cells; and (d) administering the expanded PD-1⁺ T cells to the subject.

In some embodiments, the harvested PD-1⁺ T cells are expanded according to any of the methods of expansion disclosed herein. In some embodiments, the anti-CTLA-4 antibody is tremelimumab.

Specific embodiments of the disclosure will become evident from the following more detailed description of certain embodiments and the claims.

### BRIEF DESCRIPTIONS OF THE DRAWINGS

FIG. 1 is a schematic of an exemplary PD-1 expressing T cell selection process of the disclosure and variables used in the selection process.

FIG. 2 depicts exemplary linkers connecting a capture moiety and an anti-PD-1 antibody.

FIG. 3 shows PD-1 selection results at a low biotin-to-antibody ratio (BAR) level, where the PD-1 expression level is greatest with a short linker and yield is greatest with a long linker (FIG. 3A). FIG. 3B shows the PD-1/CD8 purity and PD-1+CD8 yield, wherein purity is the percentage of cells within a selected T cell population that fall within the top 10, 20, or 30% of PD-1 expression, and yield is the percentage of T cells from the starting population that fall within the top 10, 20, or 30% of PD-1 expression.

FIG. 4 shows PD-1 selection results at a high BAR level, where the PD-1 expression level is greatest with a short linker and yield is greatest with a long linker (FIG. 4A). FIG. 4B shows the PD-1/CD8 purity and PD-1⁺ CD8 yield.

FIG. 5A shows a design of experiment display showing the impact of cell concentration, anti-PD-1 antibody concentration, and BAR on PD-1 expression levels, yield, PD-1 purity, and cell viability. FIG. 5B shows representative results of a design of experiment model fit of PD-1 expression selection data that include cell concentration, PD-1 antibody concentration, and BAR levels. Low BAR resulted in a high PD-1 expression level and low yield, and high BAR resulted in low PD-1 expression level and high yield. Low PD-1 antibody concentration resulted in high PD-1 expression level and low yield, and high PD-1 antibody concentration resulted in low PD-1 expression level and high yield. Cell concentration did not affect selection outcome within the tested range.

FIG. 6 shows PD-1 selection results when anti-biotin microbead concentration is changed. FIG. 6A shows pre-selection and post-selection numbers with 50 μL, 100 μL, and 200 μL of microbeads per mL of cell suspension. FIG. 6B shows the PD-1/CD8 purity (left) and PD-1⁺ CD8 yield (right) with 50 μL, 100 μL, and 200 μL of microbeads per mL of cell suspension.

FIG. 7 shows a flow chart illustrating the two paths of flow rates through a magnetic field and the effects shown on PD-1 expression levels.

FIG. 8 shows PD-1 expression in CD4+ and CD8+ T cells in an experiment in which total MEDI0680 (anti-PD-1 antibody) concentration was held at 5 μg/mL and magnetic bead concentration was varied between 5, 10, and 100 μL/mL.

FIG. 9 is a schematic showing an experiment in which the flow rate of a bead mixture through a magnetic field was adjusted along with an increase in magnetic field intensity.

FIG. 10 shows results of PD-1 selection at a standard flow rate and a slow flow rate with stronger magnetic field intensity for both CD4+ T cells and CD8+ T cells.

FIG. 11 shows the effect of anti-PD-1 antibody concentration on PD-1 expression level of selected T cells and overall yield. The response profile of overall yield and PD-1 expression level of isolated cells does not change relative to the K_{d} values of different anti-PD-1 antibodies. The only relative change is the position at x-axis where the effects enter plateaued phase.

FIG. 12 shows that biotinylated MEDI0680 (MEDI3097) with an average BAR of 1.9 is hetergenous and contains antibody molecules with 0, 1, 2, 3, 4, or 5 capture moiety biotin.

FIG. 13 shows the use of LO115 anti-PD-1 antibody concentration to control the PD-1 expression level of selected cells. As LO115 concentration decreases from 0.5 μg/mL to 0.05 μg/mL, the PD-1 expression level of selected cells increases.

FIG. 14 shows how the combination of MEDI3097 and unmodified MEDI0680 change PD-1 expression of selected cells. As the ratio of biotinylated to unmodified MEDI0680 decreases, the PD-1 expression level of selected cells increases.

FIG. 15 shows how the combination of biotinylated LO115 and unmodified LO115 change PD-1 expression of selected cells. As the ratio of biotinylated to unmodified LO115 decreases, the PD-1 expression level of selected cells increases.

FIG. 16 is a timeline of an exemplary priming and expansion protocol.

FIG. 17 shows results of PD-1 expansion using varying concentrations of α-inducible costimulator (ICOS) to prime the culture plate.

FIG. 18 shows comparative results of PD-1 expansion using 1 μg/mL OKT3 and anti-ICOS antibody (α-ICOS) to prime a plate versus TetAb, where manual priming showed greater efficacy of rapid expansion.

FIG. 19 shows cell proliferation of PD-1 expressing cells for varying concentrations of α-ICOS and α-ICOS clones where α-ICOS and α-ICOS clone #140 show similar proliferation efficiency.

FIG. 20A shows results measured in fold expansion of PD-1 expressing cells at Day 13 after either Bezafibrate or Luperox treatments for varying starting points and durations.

FIG. 20B shows a schematic of the seven combinations of starting points and durations tested.

FIG. 21A shows results measured in fold expansion of PD-1 expressing cells at Day 13 after either no treatment, Bezafibrate, varying concentrations of Luperox, or Bezafibrate and Luperox treatment. FIG. 21B shows a schematic of the seven combinations of chemical, concentration, and duration tested.

FIG. 22 shows results measured in fold expansion of PD-1 expressing cells at Day 13 for the 6-well optimal reseeding process.

FIG. 23 shows results measured in fold expansion of PD-1 expressing cells at Day 13 for the multidisciplinary analysis.

FIG. 24 shows results measured in fold expansion of PD-1 expressing cells at Day 13 for the G-Rex 100M full-scale expansion.

FIG. 25 shows comparison of fold expansion results for 6-well expansion and full-scale expansion. Using both methods, the combination of Luperox and the α-ICOS clone #140 showed greatest fold expansion at Day 15.

FIG. 26 is a timeline schematic depicting an exemplary reseeding culture expansion and a full-scale culture expansion of PD-1 expressing T cells.

FIG. 27 shows expansion results for α-41BB (α-CD137) coating at 0.04, 0.2, and 1.0 μg/mL.

FIG. 28 shows expansion results for α-PD-1 coating at 0.6, 3.0, and 15 μg/mL.

FIG. 29 shows expansion results for α-LAG3 coating at 0.2, 1.0, and 5 μg/mL.

FIG. 30A shows expansion results for α-TIM3 (62GL) coating at 20, 100, and 500 μg/mL. FIG. 30B shows expansion results for α-TIM3 (F9S) coating at 0.2, 1.0, and 5 μg/mL.

FIG. 31A shows expansion results for α-TIGIT (1170) coating at 0.2, 1.0, and 5 μg/mL. FIG. 31B shows expansion results for α-TIGIT (1182) coating at 0.2, 1.0, and 5 μg/mL. FIG. 31C shows expansion results for α-TIGIT (1170) coating at 5 μg/mL with α-CD226-A at 0.4, 2.0, and 10 μg/mL. FIG. 31D shows expansion results for α-TIGIT (1170) coating at 5 μg/mL with α-CD226-B at 0.4, 2.0, and 10 μg/mL. FIG. 31E shows expansion results for α-TIGIT (1170) coating at 5 μg/mL with α-CD226-C at 0.4, 2.0, and 10 μg/mL. FIG. 31F shows expansion results for α-TIGIT (1182) coating at 5 μg/mL with α-CD226-A at 0.4, 2.0, and 10 μg/mL. FIG. 31G shows expansion results for α-TIGIT (1182) coating at 5 μg/mL with α-CD226-B at 0.4, 2.0, and 10 μg/mL. FIG. 31H shows expansion results for α-TIGIT (1170) coating at 5 μg/mL with α-CD226-C at 0.4, 2.0, and 10 μg/mL.

FIG. 32 shows expansion results for α-ICOS coating at 1 μg/mL with or without soluble PD-1 at 0.6 and 3 μg/mL or coated α-41BB at 0.04 and 0.2 μg/mL.

FIG. 33A shows results of enrichment of PD-1 high expresser T cells from melanoma patient PBMC. FIG. 33B shows expansion of PD-1 high expresser T cells without the use of feeder cells or human serum. FIG. 33C shows results of testing where cytotoxic T cell persistence is maintained in vivo through the use of ICOS, Luperox, and cytokines.

FIG. 34 is a schematic of personalized T cell therapy options.

FIG. 35 shows expansion results for α-OX40 coating.

FIG. 36A shows proliferation of CD4 cells after incubation for four days with 4:1, 2:1, 1:1, 1:2, 1:4, 1:8, or 1:16 M280 anti-ICOS or anti-CD3 coated tosylactivated beads. FIG. 36B shows proliferation of CD4 cells after incubation for four days with 4:1, 2:1, 1:1, 1:2, 1:4, 1:8, or 1:16 M450 anti-ICOS or anti-CD3 coated tosylactivated beads.

FIG. 37A shows proliferation of CD8 cells after incubation for four days with 4:1, 2:1, 1:1, 1:2, 1:4, 1:8, or 1:16 M280 anti-ICOS or anti-CD3 coated tosylactivated beads. FIG. 37B shows proliferation of CD8 cells after incubation for four days with 4:1, 2:1, 1:1, 1:2, 1:4, 1:8, or 1:16 M450 anti-ICOS or anti-CD3 coated tosylactivated beads.

FIG. 38A shows PD-1 expression on CD4 cells after incubation for four days with 4:1, 2:1, 1:1, 1:2, 1:4, 1:8, or 1:16 M280 anti-ICOS or anti-CD3 coated tosylactivated beads. FIG. 38B shows PD-1 expression on CD4 cells after incubation for four days with 4:1, 2:1, 1:1, 1:2, 1:4, 1:8, or 1:16 M450 anti-ICOS or anti-CD3 coated tosylactivated beads.

FIG. 39A shows PD-1 expression on CD8 cells after incubation for four days with 4:1, 2:1, 1:1, 1:2, 1:4, 1:8, or 1:16 M280 anti-ICOS or anti-CD3 coated tosylactivated beads. FIG. 39B shows PD-1 expression on CD8 cells after incubation for four days with 4:1, 2:1, 1:1, 1:2, 1:4, 1:8, or 1:16 anti-ICOS or anti-CD3 coated M450 tosylactivated beads.

FIG. 40A shows a schematic for anti-ICOS treatment during T cell expansion. This is a timeline for the stimulation by anti-ICOS in early time-course manner (Day 0-4) in PD-1-CTL REP. FIG. 40B show that ICOS agonism is sufficient in the first 4 days of stimulation to prime T cells for expansion.

FIG. 41A shows a schematic for anti-ICOS treatment during T cell expansion. This is a timeline for the stimulation by anti-ICOS in early time-course manner (Day 4-10) in PD-1-CTL REP. FIG. 41B shows that 4 days of priming with anti-ICOS results in the sufficient proliferation.

FIG. 42 shows the requirements for IL2, and details a comparison of relative fold expansion at Day 13 by averaging all 7 samples (3 LPs and 4 Bloods).

FIG. 43A shows CD25 expression on CD4 cells after incubation for four days with 4:1, 2:1, 1:1, 1:2, 1:4, 1:8, or 1:16 M280 anti-ICOS or anti-CD3 coated tosylactivated beads. FIG. 43B shows CD25 expression on CD4 cells after incubation for four days with 4:1, 2:1, 1:1, 1:2, 1:4, 1:8, or 1:16 M450 anti-ICOS or anti-CD3 coated tosylactivated beads.

FIG. 44A shows CD25 expression on CD8 cells after incubation for four days with 4:1, 2:1, 1:1, 1:2, 1:4, 1:8, or 1:16 M280 anti-ICOS or anti-CD3 coated tosylactivated beads. FIG. 44B shows CD25 expression on CD8 cells after incubation for four days with 4:1, 2:1, 1:1, 1:2, 1:4, 1:8, or 1:16 anti-ICOS or anti-CD3 coated M450 tosylactivated beads.

FIG. 45A shows fold expansion of PD-1⁺ T cells after standard rapid expansion protocol (REP) as described in Dudley et al., 2003, J. Immunother. 26(4): 332-42, or optimized REP disclosed in Example 11. FIG. 45B shows percent T-cell ratios at day 14 after standard REP or optimized REP as compared with ratios at day 0.

FIG. 46 shows mitochondrial mass and glucose consumption in CD4 and CD8 T cells with or without ICOS treatment.

FIG. 47 shows expression of telomerase reverse transcriptase (TERT) after treatment with CD3/CD28, CD3/CD28+ anti-ICOS, CD3/CD28+ anti-ICOS+leucine, or TetAb.

FIG. 48 shows T cell phenotype following various agonist treatments. NIP=Control antibody, OKT3=anti-CD3 antibody, Lup=Luperox, Tem=effector-memory cell phenotype, Temra=Tem cells that express CD45RA, Tcm=central-memory cell phenotype, Tn=naïve T cell phenotype.

FIG. 49 shows percent antigen-specific T cell survival following agonist treatments. NIP=Control antibody, OKT3=anti-CD3 antibody, Lup=Luperox.

FIG. 50 shows the effect of ICOS agonism on cytotoxic activity of expanded PD-1 T cells. Only those cells described as “ICOS primed PD-1CTL w IL-2” showed strong CMV/EBV/FluA specific cytotoxic activity at high E/T ratio 30. PD-1 CTL primed with only OKT3/sCD28 or primed with ICOS without IL-2 didn't show cytotoxic activity even if they had a similar frequency of CMV/EBV/FluA-specific T cells with ICOS/IL-2 primed PD-1 CTL.

FIGS. 51A and 51B show CD8⁺ IL-2 induction after treatment with 1 μg/mL monoclonal antibodies (anti-CD3 (aCD3), anti-OX40 (MEDI0562), or MEDI0562+ anti-CD3), IgG1, or Staphyloccoccal Enterotoxin B (SEB).

FIGS. 52A and 52B show TILs in multiple type of cancers express high level of PD-1.

FIG. 53 shows the effects of tremelimumab. In FIG. 53A, both squares and circles were tremelimumab treated. The circles show a group of patients with less than 1.5 fold changes (<1.5 fold) of PD-1 expression. The squares show patients that had >1.5 fold increase of PD-1 expression after tremelimumab treatment. The high responder group (squares) showed better overall survival as compared to the low responder group (circles). This therefore indicates that tremelimumab treatment can induce PD-1 expression 9-10 days after treatment. FIG. 53B shows that tremelimumab induced PD-1 expression correlates with improved overall survival (OS).

FIG. 54A shows that durvalumab/tremelimumab combination treatment induces PD-1+ T cells in blood. FIG. 54B shows that durvalumab/tremelimumab combination treatment-induced PD-1 expression correlated with improved OS; Study 11 SCCHN: Q4W Durva 20 mg/Kg+Treme 1 mg/kg; PBMC flow data n=35.

FIG. 55 shows a schematic of a clinical trial for treatment of melanoma using ex vivo capture of PD-1⁺ T cells following mobilized with Durva/Treme. Treme=tremelimumab, PBMC=peripheral blood mononuclear cells, IO=Immunotherapy, TIL=tumor infiltrating lymphocytes.

FIG. 56 shows T cell effector function as it relates to cell surface phenotype, e.g., expression of factors such as PD-1, T cell immunoreceptor with Ig and ITIM domains (TIGIT), lymphocyte-activation gene 3 (LAG-3), TIM-3, and CD200R.

FIG. 57 shows the results from biotinylated MEDI0680 using PEG4 linker isolated T cells.

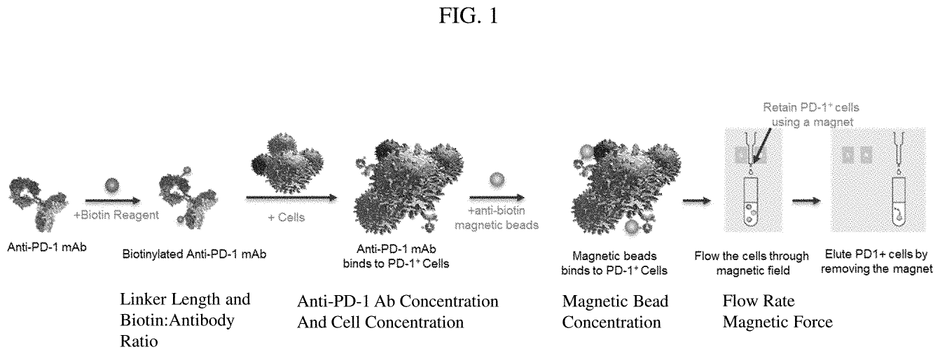

### DETAILED DESCRIPTION

The disclosure provides methods for the selection and isolation of T cells expressing PD-1, and for selecting a PD-1 expression level of the isolated PD-1 expressing T cells. In the methods of the disclosure, the PD-1 expression level of the PD-1 expressing T cells may be selected by adjusting one or more parameters or variables of the disclosed methods. The disclosure also provides methods of large scale expansion of such selected and isolated PD-1 expressing T cells. The disclosure further provides methods for treating a subject comprising administering selected and isolated PD-1 expressing T cells to the subject. The disclosure further provides a magnetic bead-based capture system to isolate T cells from peripheral blood expressing different levels of PD-1 quickly enough to permit their expansion ex vivo.

The methods disclosed herein can also be used, e.g., to select tumor reactive T cells from T cells isolated from tumors (TIL). In additional aspects, the tumor reactive T cells can be used to identify individualized or shared antigens that can also be used for T cell receptor therapy (TCRT) or chimeric antibody receptor therapt (CART).

As utilized in accordance with the present disclosure, the following terms, unless otherwise indicated, shall be understood to have the following meanings. Unless otherwise required by context, singular terms shall include pluralities and plural terms shall include the singular.

The term “patient” or “subject,” as used herein, includes human and animal subjects.

A “disorder” is any condition that would benefit from treatment using the enriched and expanded T cells of the disclosed method. “Disorder” and “condition” are used interchangeably herein, and include chronic and acute disorders or diseases, including those pathological conditions that predispose a patient to the disorder in question.

As used herein, “enrich” or “enrichment” means to increase the percentage of PD-1⁺ T cells by at least 2-fold if the percentage of PD-1⁺ T cells of starting cells is less than 10% or to increase the percentage of PD-1+ T cells to at least 50% if the percentage of PD-1⁺ T cells of starting cells is equal or higher than 10%.

The terms “treatment” or “treat,” as used herein, refer to both therapeutic treatment and prophylactic or preventative measures. Those in need of treatment include those having the disorder as well as those prone to having the disorder or those in which the disorder is to be prevented.

As used herein, the terms “anti-,” “α-,” and “a-” are used interchangeably and refer to an antibody against the target that follows the hyphen. In some instances, the hyphen is omitted. Thus, merely by way of example, “α-ICOS,” “α-ICOS,” “aICOS,” and “aICOS” refer to anti-ICOS antibody, and “α-PD-1,” “α-PD-1,” “aPD-1,” and “aPD-1” refer to anti-PD-1 antibody.

The disclosure includes methods of enriching cytotoxic T cells expressing desired levels of PD-1. When referring to the amount of expression of PD-1 on or within a population of cells, expression levels may be referred to as high, intermediate, or low, and may also be referred to as bright, medium, and dim, respectively.

Programmed cell death 1 (PD-1) is a 50-55 kDa type I transmembrane receptor originally identified in a T cell line undergoing activation-induced apoptosis. PD-1 is expressed on T cells, B cells, and macrophages. The ligands for PD-1 are the B7 family members PD-L1 (B7-H1) and PD-L2 (B7-DC). PD-1 is a member of the immunoglobulin (Ig) superfamily that contains a single Ig V-like domain in its extracellular region. The PD-1 cytoplasmic domain contains two tyrosine residues, with the most membrane-proximal tyrosine residue (VAYEEL in the murine PD-1) located within an immuno-receptor tyrosine-based inhibitory motif (ITIM). The presence of an ITIM on PD-1 indicates that this molecule functions to attenuate antigen receptor signaling by recruitment of cytoplasmic phosphatases. Human and murine PD-1 proteins share about 60% amino acid sequence identity with conservation of four potential N-glycosylation sites, and residues that define the Ig-V domain. The ITIM in the cytoplasmic region and the ITIM-like motif surrounding the carboxy-terminal tyrosine (TEYATI in human and mouse) are also conserved between human and murine orthologues. PD-1, which is also known as cluster of differentiation 279 or CD279, performs as an immune checkpoint, as it promotes apoptosis in antigen specific T cells in lymph nodes but also inhibits apoptosis in regulatory T cells.

In some embodiments, PD-1 expression levels are measured or assessed by using PD-1/CD8 purity values. PD-1/CD8 purity (also referred to herein as PD-1CD8 purity or CD8PD-1 purity) is defined as the percentage of PD-1 expressing CD8+ T cells selected or isolated according to the methods of the disclosure that fall within a certain percentage of PD-1 expressing T cells. For example, PD-1/CD8 Purity 10 is the percentage of selected PD-1 expressing CD8+ T cells that fall within the highest 10 percent of PD-1 expression. In some embodiments, PD-1/CD8 Purity 10 is a measure of high PD-1 expression, and the percentage of PD-1/CD8 Purity 10 cells is a measure of the percentage of isolated PD-1 expressing T cells with high PD-1 expression.

In another example, PD-1/CD8 Purity 20 is the percentage of selected PD-1 expressing CD8+ T cells that fall within the highest 20 percent of PD-1 expression. In some embodiments, the percentage of PD-1/CD8 Purity 20 cells is a measure of the percentage of isolated PD-1 expressing T cells with intermediate PD-1 expression.

In another example, PD-1/CD8 Purity 30 is the percentage of selected PD-1 expressing CD8+ T cells that fall within the highest 30 percent of PD-1 expression. In some embodiments, the percentage of PD-1/CD8 Purity 30 cells is a measure of the percentage of isolated PD-1 expressing T cells with low PD-1 expression.

In some embodiments, the terms “high expression,” “high expresser,” or “high expressing,” as used herein, refer to a subset of isolated cells that are positive for expression of the indicated cell marker, and which produce a higher signal for the indicated cell marker using one or more of the following methods (e.g., FACS, flow cytometry, immunofluorescence assays, or microscopy) than other cells that are positive for expression of the indicated cell marker. Thus, in some embodiments, cells with “high PD-1 expression” refers to cells positive for PD-1, and which produce a higher PD-1 signal compared to other cells in the population as measured by, for example, flow cytometry. For example, cells with a “high” level of expression of the indicated cell marker may produce a higher signal for the marker than about 50%, about 60%, about 70%, about 80%, about 90%, or about 95%, or a range of any two of the foregoing values, of the other cells that are positive for expression of the indicated cell marker.

In some embodiments, the terms “low expression,” “low expresser,” or “low expressing,” as used herein, refer to a subset of isolated cells that are positive for expression of the indicated cell marker, and which produce a low signal for the indicated cell marker using one or more of the following methods (e.g., FACS, flow cytometry, immunofluorescence assays, or microscopy) than other cells that are positive for expression of the indicated cell marker. Thus, in some embodiments, cells with “low PD-1 expression” refers to cells positive for PD-1, and which produce a lower PD-1 signal compared to other cells in the population as measured by, for example, flow cytometry. For example, cells with a “low” level of expression of the indicated cell marker may produce a lower signal for the marker than about 50%, about 60%, about 70%, about 80%, about 90%, or about 95%, or a range of any two of the foregoing values, of the other cells that are positive for expression of the indicated cell marker.

In some embodiments, the terms “intermediate expression,” “intermediate expresser,” or “intermediate expressing,” as used herein, refer to a subset of isolated cells that are positive for expression of the indicated cell marker, and which a signal for the indicated cell marker somewhere between high expressing cells and low expressing cells using one or more of the following methods (e.g., FACS, flow cytometry, immunofluorescence assays, or microscopy). Thus, in some embodiments, cells with “intermediate PD-1 expression” refers to cells positive for PD-1, and which produce a lower PD-1 signal compared to some cells in the population and a higher PD-1 signal compared to other cells in the population as measured by, for example, flow cytometry.

In one aspect, the disclosure provides methods of isolating T cells expressing programmed cell death 1 (PD-1) from a cell population, comprising:

(a) contacting the cell population with an amount of an anti-PD-1 antibody to produce an antibody-cell mixture, wherein the anti-PD-1 antibody comprises a capture moiety, and wherein the capture moiety is connected to the anti-PD-1 antibody via a linker;

(b) contacting the antibody-cell mixture with an amount of magnetic beads, wherein the magnetic beads are capable of specifically binding the capture moiety on the anti-PD-1 antibody to produce a bead mixture;

(c) passing the bead mixture through a magnetic field to isolate the magnetic beads and PD-1 expressing T cells bound thereto from the bead mixture; and

(d) eluting the PD-1 expressing T cells from the magnetic field to isolate T cells expressing PD-1.

In another aspect, the disclosure provides methods further comprising selecting a PD-1 expression level of the T cells expressing PD-1 isolated in step (d) by adjusting one or more of:

(i) the concentration of the anti-PD-1 antibody in the antibody-cell mixture;

(ii) the length of the linker;

(iii) the stoichiometric ratio of capture moiety to anti-PD-1 antibody (CAR);

(iv) the ratio of anti-PD-1 antibody comprising a capture moiety to unmodified anti-PD-1 antibody;

(v) the temperature at which either step (a) and/or step (b) is carried out;

(vi) the antibody-cell mixture and/or in the bead mixture;

(vii) the concentration of magnetic beads in the bead mixture;

(viii) the flow rate at which the bead mixture is passed through the magnetic field;

(ix) the length of time between the production of the antibody-cell mixture and step (b);

(x) the length of time between production of the bead mixture and step (c); or

(xi) the magnetic field strength.

T cells can be enriched according to the methods disclosed herein from any T cell containing sample or cell population, including, for example leukapheresis products obtained from healthy or diseased individuals. In some embodiments, T cells are obtained according to the methods disclosed herein from leukapheresis starting products obtained from subjects with cancer.

In some embodiments, the T cells are CD8+ T cells. In other embodiments, the T cells are CD4+ T cells. As used herein, the term “T cells” refers to T lymphocytes, which are a type of white blood cell that plays a central role in cell-mediated immunity. T cells can be distinguished from other lymphocytes, such as B cells and natural killer cells, by the presence of a T cell receptor on the cell surface. The several subsets of T cells each have a distinct function. The majority of human T cells rearrange their alpha and beta chains on the cell receptor and are termed alpha beta T cells (αβ T cells) and are part of the adaptive immune system. There are two major types of T cells, helper T cells (CD4+) and cytotoxic T cells (CD8+). Most cytotoxic T cells express T-cell receptors (TCRs) which recognize a specific antigen bound to class I MHC molecules.

T cells can be selected from bulk populations of peripheral blood mononuclear cells (PBMCs) from a sample of peripheral blood of a patient by any suitable method known in the art. Such methods of obtaining a bulk population of PBMCs may include, but are not limited to, a blood draw and/or a leukapheresis. The peripheral blood can be taken from healthy or diseased individuals. The bulk population of PBMCs obtained from a peripheral blood sample may comprise T cells, including tumor-reactive T cells (TIL) and marrow infiltrating lymphocytes (MIL). In other aspects, T cells can be selected from tumor draining lymphnodes, bone marrow, or disaggregated tumor tissue.

Non-limiting examples of T cells that express PD-1 include T cells characterized by the following marker combinations: CD8+PD-1+; PD-1+TIM-3+; PD-1+CD27+; CD8+PD-1 high expressers; CD8+PD-1+TIM-3+; CD8+PD-1+CD27 high expressers; CD8+PD-1+CD27+; CD8+PD-1+TIM-3-; CD8+PD-1+CD27-; CD4+PD-1+; CD4+PD-1hi; CD4+PD-1+TIM-3+; CD4+PD-1+CD27 high expressers; CD4+PD-1+CD27+; CD4+PD-1+TIM-3-; and CD4+PD-1+CD27 T cells, where (+) means the cells express the marker and (−) means the cells do not express the marker. Other markers that can be expressed on CD4+ or CD8+ T cells are inducible T cell costimulator (ICOS), TIGIT, OX40, LAG-3, GITR, CTLA-4, and 41BB (CD137).

In some embodiments of the disclosure, the capture moiety is biotin. As used herein, the term “capture moiety” refers to a chemical moiety attached to a molecule that can be used to capture the molecule, for example, through interaction with another chemical moiety, for purposes such as affinity purification, immunoprecipitation, or co-immunoprecipitation. For example, a biotin capture moiety can be used in conjunction with a streptavidin column to separate, isolate, or affinity purify the molecule comprising the biotin moiety. A poly-histidine tag (His-tag, 6×His-tag, hexa histidine-tag, or His6-tag) is a capture moiety comprising at least six histidine amino acid residues that can be used to capture a His-tagged molecule because the string of histidine residues binds to several types of immobilized metal ions, including nickel, cobalt, and copper, under specific buffer conditions. In addition, anti-His-tag antibodies are commercially available for use in methods involving His-tagged proteins. Any protein for which an antibody specific for that protein exists can comprise a capture moiety. Other examples of capture moieties include a hemagglutinin (HA) tag, streptavidin-binding peptide, calmodulin-binding peptide, S-peptide, or chitin-binding domain.

In the methods of the disclosure, the capture moiety is connected to the anti-PD-1 antibody through a linker. As used herein, “linker” refers to any chemical linkage connecting two chemical entities, such as a capture moiety and an anti-PD-1 antibody. In some embodiments, the linker between the capture moiety and the anti-PD-1 antibody is a polyethylene glycol (PEG) linker. In some embodiments, the PEG linker is between 1 and 12 monomer units in length. In other embodiments, the linker is an alkyl chain with between 1 and 10 carbon atoms. In some embodiments, the capture moiety is biotin, which is linked to the anti-PD-1 antibody using sulfo-NHS-biotin, sulfo-NHS-LC-biotin, sulfo-NHS-LC-LC-biotin, or NHS-PEG4-biotin.

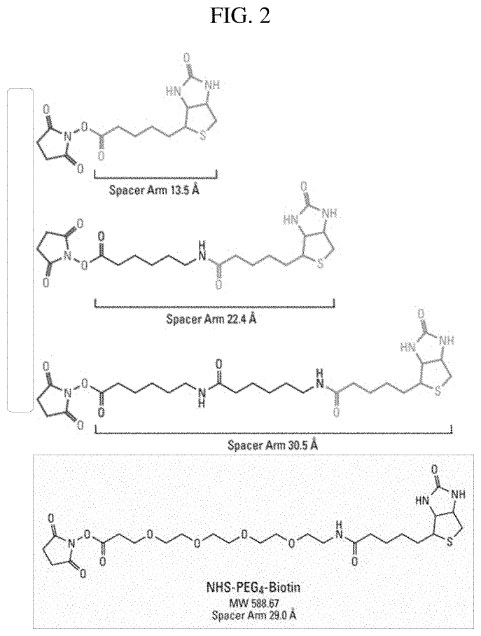

In some embodiments, the linker ranges in length from about 1 Å to about 50 Å. In some embodiments, the linker is about 1 Å, or about 2 Å, or about 3 Å, or about 4 Å, or about 5 Å, or about 10 Å, or about 15 Å, or about 20 Å, or about 25 Å, or about 30 Å, or about 35 Å, or about 40 Å, or about 45 Å, or about 50 Å in length. In other embodiments, the linker is about 13.5 Å, 22.4 Å, 29.0 Å, or 30.5 Å in length. In some embodiments, with respect to the enrichment methods disclosed herein, the term “short linker” refers to a linker of no more than about 17 Å in length; “intermediate linker” refers to a linker between about 17 and about 26 Å in length; and “long linker” refers to a linker exceeding about 26 Å in length. FIG. 2 shows exemplary linker lengths which can be used to influence selection of T cells with varying PD-1 expression levels according to methods disclosed herein.

Generally, the length of the linker used in the methods disclosed herein is negatively correlated with the PD-1 expression level of enriched T cells, and is positively correlated with the overall yield of enriched PD-1 expressing T cells. Thus, use of a short linker results in enrichment of T cells with high PD-1 expression levels and low yield of PD-1 expressing T cells, and use of a long linker results in enrichment of T cells with low PD-1 expression levels and high yield of PD-1 expressing T cells. Thus, in some embodiments, increasing the length of the linker decreases the PD-1 expression level of the isolated T cells expressing PD-1. In some embodiments, increasing the length of the linker increases the yield of isolated T cells expressing PD-1.

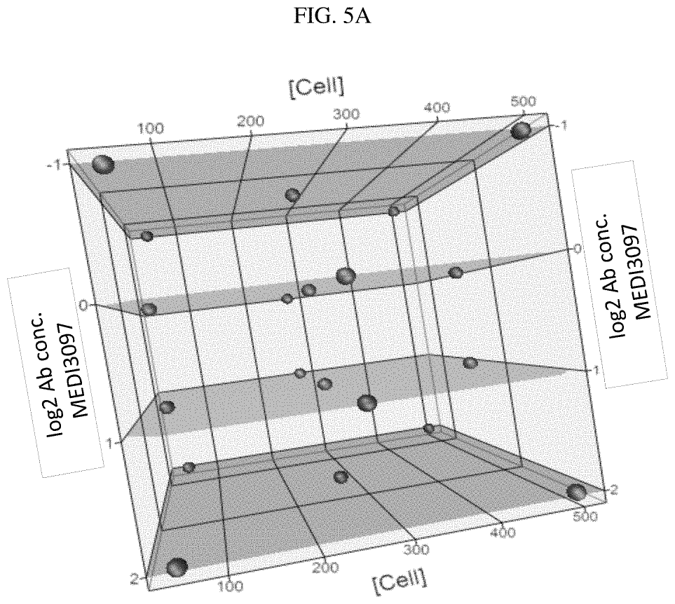

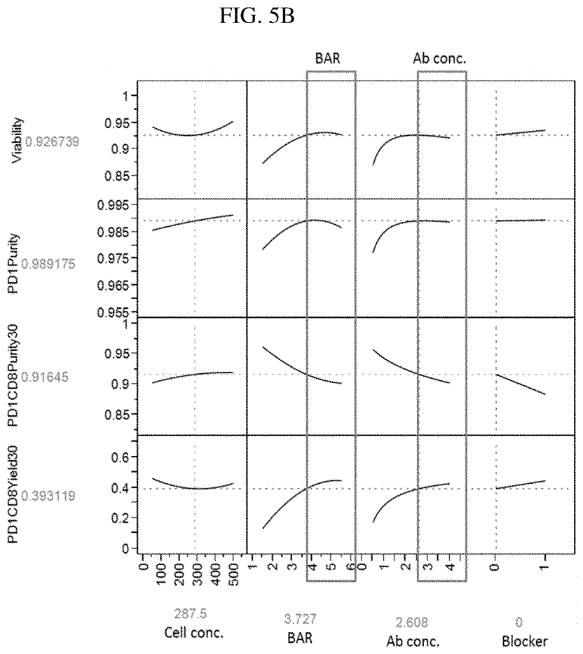

As used herein, the term “CAR” refers to the stoichiometric ratio of capture moiety concentration to antibody concentration. In some embodiments, CAR is used as a variable for selecting the PD-1 expression level and yield of PD-1 expressing T cells isolated according to the methods disclosed herein. In embodiments in which biotin is the capture moiety, CAR is also referred to as the biotin-to-antibody ratio (BAR). In some embodiments, CAR impacts the PD-1 expression levels and/or yield of PD-1 expressing cells obtained using the methods disclosed herein. Generally, in the methods disclosed herein, when CAR is decreased, the PD-1 expression levels increase and the yield of PD-1 expressing cells in the eluent decreases. Alternatively, when CAR is increased, the PD-1 expression levels decrease and the overall yield of PD-1 expressing cells in the eluent increases (see, e.g., Example 2 and FIG. 5). In some embodiments, when CAR is increased, the PD-1 expression level of isolated T cells expressing PD-1 decreases. In some embodiments, when CAR is increased, the yield of T cells expressing PD-1 increases. In some embodiments, CAR is between 1 and 8. In some embodiments, CAR is between 1 and 7.5. In other embodiments, CAR is about 1, or about 1.5, or about 2, or about 2.5, or about 3, or about 3.5, or about 4, or about 4.5, or about 5, or about 5.5, or about 6, or about 6.5, or about 7, or about 7.5. In other embodiments, CAR is about 1.7, 5.2, or 6.4.

In some embodiments, the concentration of T cells within the cell population is between 20 million cells/mL to 500 million cells/mL. In other embodiments, the concentration of T cells within the cell population is about 20 million cells/mL, or about 50 million cells/mL, or about 100 million cells/mL, or about 150 million cells/mL, or about 200 million cells/mL, or about 250 million cells/mL, or about 275 million cells/mL, or about 300 million cells/mL, or about 350 million cells/mL, or about 400 million cells/mL, or about 450 million cells/mL, or about 500 million cells/mL.

In some embodiments, the cell population is obtained from a healthy subject. In other embodiments, the cell population is obtained from a subject with cancer.

In some embodiments, the concentration of magnetic beads is between about 1 μL per 1×10⁷ cells and about 100 μL per 1×10⁷ cells. In other embodiments, the concentration of magnetic beads is between about 1 μL per 1×10⁷ cells and 30 μL per 1×10⁷ cells. In other embodiments, the concentration of magnetic beads is about 5 μL per 1×10⁷ cells, or about 10 μL per 1×10⁷ cells, or about 20 μL per 1×10⁷ cells, or about 100 μL per 1×10⁷ cells. In general, in the methods disclosed herein, PD-1 expression levels in the eluent negatively correlate with magnetic bead concentration, whereas yield of PD-1 expressing cells in the eluent positively correlate with magnetic bead concentration. Thus, in some embodiments, increasing the magnetic bead concentration decreases the PD-1 expression level of isolated T cells expressing PD-1. In some embodiments, increasing the magnetic bead concentration increases the yield of isolated T cells expressing PD-1.

Any anti-PD-1 antibody can be used in the methods disclosed herein. Non-limiting examples of PD-1 antibodies that could be used in the methods disclosed herein can be found, for example, in Agata et al., 1996, Int. Immunol. 8(5): 765-72, and in U.S. Pat. Nos. 7,488,802 and 8,088,905, all of which are incorporated herein by reference in their entireties.

In some embodiments, the anti-PD-1 antibody concentration in the antibody-cell mixture is between 0.1 μg/mL and 10 μg/mL. In other embodiments, the antibody concentration in the antibody-cell mixture is between 0.5 μg/mL and 5 μg/mL.

In some embodiments, the anti-PD-1 antibody is LO115. In certain aspects, LO115 comprises a first light chain CDR having the sequence SASSKHTNLYWSRHMYWY, a second light chain CDR having the sequence LTSNRAT, and a third light chain CDR having the sequence QQWSSNP; and a first heavy chain CDR having the sequence GFTFSDYGMH, a second heavy chain CDR having the sequence YISSGSYTIYSADSVKG, and a third heavy chain CDR having the sequence RAPNSFYEYYFDY. In aspects disclosed herein, LO115 comprises a light chain comprising the amino acid sequence QIVLTQSPATLSLSPGERATLSCSASSKHTNLYWSRHMYWYQQKPGQAPRLLIYLTSNR ATGIPARFSGSGSGTDFTLTISSLEPEDFAVYYCQQWSSNPFTFGQGTKLEIKRTVAAPSV FIFPPSDEQLKSGTASVVCLLNNFYPREAKVQWKVDNALQSGNSQESVTEQDSKDSTYS LSSTLTLSKADYEKHK; and a heavy chain comprising the amino acid sequence EVQLVESGGGLVQPGGSLRLSCAASGFTFSDYGMHWVRQAPGKGLEWVAYISSGSYTI YSADSVKGRFTISRDNAKNSLYLQMNSLRAEDTAVYYCARRAPNSFYEYYFDYWGQG TTVTVSSASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKRVEPKSCDKTHTCPPCP APEFEGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPASIEKTISKAKGQPREPQ VCTLPPSREEMTKNQVSLSCAVKGFYPSDIAVEWESNGQPENNYKTTPPVLDSDGSFFL VSKLTVDKSRWQQGNVFSCSVMHEALHNHYTQKSLSLSPGK.

In some embodiments, the anti-PD-1 antibody is LO115, and the antibody concentration is between 0.01 μg/mL and 1 μg/mL.

In other embodiments, the anti-PD-1 antibody is MEDI0680. As used herein, biotinylated MEDI0680 is MEDI3097. In certain aspects, MEDI0680 comprises a first light chain CDR having the sequence SASSSVSYMY, a second light chain CDR having the sequence LTSNRAT, and a third light chain CDR having the sequence QQWSSNPFT; and a first heavy chain CDR having the sequence GFTFSDYGMH, a second heavy chain CDR having the sequence YISSGSYTIYSADSVKG, and a third heavy chain CDR having the sequence RGYGSFYEYYFD. In a further aspect, MEDI0680 comprises a light chain variable region comprising the amino acid sequence QIVLTQSPATLSLSPGERAT LSCSASSSVS YMYWYQQKPGQAPRLLIYLTSNRATGIPARFSGSGSGTDYSS TLTISSLEPEDFAVYYCQQWSSNPFTFGQGTKLEIK; and a heavy chain variable region comprising the amino acid sequence EVQLVESGGGLVQPGGSLRLSCAASGFTFS DYGMHWVRQAPGKGLEWVAYISSGSYTIYSADSVKGRFTISRDNAKNTLY LQMSSLRAEDTAVYYCARRGYGSFYEYYFDYWGQGTTVTVSS.

In further embodiments, the anti-PD-1 antibody is MEDI0680, and the antibody concentration is between 0.5 μg/mL and 5 μg/mL.

In general, in the methods disclosed herein, PD-1 expression levels on selected T cells are inversely correlated with anti-PD-1 antibody concentration, and yield of PD-1 expressing cells positively correlates with anti-PD-1 antibody concentration. Thus, in some embodiments, increasing the antibody concentration in the antibody-cell mixture decreases the PD-1 expression level of isolated T cells expressing PD-1. In other embodiments, increasing the antibody concentration in the antibody-cell mixture increases the yield of isolated T cells expressing PD-1.

In some embodiments of the methods disclosed herein, the mixture of cells, antibodies, and magnetic beads, which contains a population of cells bound by anti-PD-1 antibodies, which are in turn bound to the magnetic beads via the capture moiety, is passed through a magnetic field more than one time. In some embodiments, the bead mixture is first passed through the magnetic field at a high flow rate, and the PD-1 expressing T cells eluted from the magnetic beads isolated from the high flow rate passage have a high PD-1 expression level; next, the primary negative fraction of the bead mixture remaining after elution of the high PD-1 expressing cells from the bead mixture is passed through the magnetic field a second time at an intermediate flow rate, producing a secondary fraction of captured cells that comprises T cells with an intermediate PD-1 expression level; and next, the secondary negative fraction of the bead mixture remaining after elution of the intermediate PD-1 expressing cells is passed through the magnetic field a third time at a low flow rate, producing a tertiary fraction of captured cells that comprises T cells with a low PD-1 expression level. In some embodiments, the bead mixture is first passed through the magnetic field at a low flow rate, producing a captured cell fraction and a discarded fraction of the bead mixture; next, the captured cell fraction is passed through the magnetic field a second time at an intermediate flow rate, producing a secondary fraction of captured cells and a negative fraction, wherein the negative fraction comprises T cells with a low PD-1 expression level; and next, the secondary fraction of captured cells is passed through the magnetic field a third time at a high flow rate, producing a tertiary fraction of captured cells and a second negative fraction, wherein the second negative fraction comprises T cells with an intermediate PD-1 expression level and the tertiary fraction of captured cells comprises T cells with a high PD-1 expression level.

Thus, in some embodiments of the methods disclosed herein, the steps of passing the bead mixture through a magnetic field and eluting the PD-1 expressing T cells from the magnetic field comprise:

(a) passing the bead mixture through the magnetic field at a high flow rate and/or low magnetic field intensity;

(b) eluting PD-1 expressing T cells from the magnetic field to isolate T cells having a high PD-1 expression level;

(c) passing a primary negative fraction of the bead mixture remaining after elution of the T cells having a high PD-1 expression level through the magnetic field at an intermediate flow rate and/or intermediate magnetic field intensity;

(d) eluting PD-1 expressing T cells from the magnetic field to isolate T cells having an intermediate PD-1 expression level;

(e) passing a secondary negative fraction of the bead mixture remaining after elution of the T cells having an intermediate PD-1 expression level through the magnetic field at a low flow rate and/or high magnetic field intensity; and

(f) eluting PD-1 expressing T cells from the magnetic field to isolate T cells having a low PD-1 expression level.

In other embodiments of the methods disclosed herein, the steps of passing the bead mixture through a magnetic field and eluting the PD-1 expressing T cells from the magnetic field comprise:

(a) passing the bead mixture through the magnetic field at a low flow rate and/or high magnetic field intensity to produce a first captured bead mixture fraction and a discarded bead mixture fraction;

(b) eluting the first captured bead mixture fraction from the magnetic field;

(c) passing the first captured bead mixture fraction through the magnetic field at an intermediate flow rate and/or intermediate magnetic field intensity to produce a secondary captured bead mixture fraction and a first negative bead mixture fraction, wherein the first negative bead mixture fraction comprises T cells having a low PD-1 expression level;

(d) passing the secondary captured bead mixture fraction through the magnetic field at a high flow rate and/or low magnetic filed intensity to produce a tertiary captured bead mixture fraction and a second negative bead mixture fraction, wherein the tertiary captured bead mixture fraction comprises T cells having a high PD-1 expression level and the second negative bead mixture fraction comprises T cells having an intermediate PD-1 expression level.

In some embodiments, the high flow rate ranges from 11.0 cm/min to 20.0 cm/min. In some embodiments, the intermediate flow rate ranges from 4.0 cm/min to 8.0 cm/min. In some embodiments, the low flow rate ranges from 1.0 cm/min to 3.0 cm/min.

In some embodiments, the force applied by the magnetic field is varied to select for higher or lower expression of PD-1 or higher or lower yield of PD-1 expressing cells in the T cell population isolated according to methods of the disclosure. In some embodiments, the magnetic field intensity is varied along with the flow rate during consecutive passes of the bead mixture through the magnetic field in order to obtain low, intermediate, and high PD-1 expressing T cells in different passes. For example, in some embodiments, the bead mixture is first passed through the magnetic field at a high flow rate, wherein the PD-1 expressing T cells eluted from the magnetic beads isolated from the high flow rate passage have a high PD-1 expression level; next, the primary negative fraction of the bead mixture remaining after elution of the high PD-1 expressing cells from the bead mixture is passed through the magnetic field a second time at an intermediate flow rate, producing a secondary fraction of captured cells that comprises T cells with an intermediate PD-1 expression level; and next, the secondary negative fraction of the bead mixture remaining after elution of the intermediate PD-1 expressing cells is passed through the magnetic field a third time at a low flow rate but with a stronger magnetic field intensity as compared to the first two passes, producing a tertiary fraction of captured cells that comprises T cells with a low PD-1 expression level.

In some embodiments of the methods disclosed herein, the PD-1 expression level of the isolated T cells expressing PD-1 is adjusted according to Formula I:

$$N ( label ) = \frac{N * α * {[ Ab ]}_{i}}{{K}_{d} + {[ Ab ]}_{i}} ,$$

wherein:

N(label) is the number of expected label molecules on the T cells expressing PD-1, which is negatively correlated with PD-1 expression level of isolated T cells;

N is the number of PD-1 antigen-binding sites on the T cells expressing PD-1;

[Ab]ᵢ is the initial concentration of the anti-PD-1 antibody in the antibody-cell mixture;

α is the ratio of anti-PD-1 antibody with accessible capture moiety to the total anti-PD-1 antibody; and

K_{d} is the dissociation constant of the anti-PD-1 antibody at the incubation temperature, i.e., the ratio of K_{off}/Kₒₙ at incubation temperature.

In some embodiments, the parameter a in Formula I above is adjusted by changing or adjusting one or more of:

(i) the length of the linker;

(ii) the stoichiometric ratio of capture moiety to anti-PD-1 antibody (CAR); or

(iii) the ratio of anti-PD-1 antibody comprising a capture moiety to unmodified anti-PD-1 antibody (which can be the same as the anti-PD-1 antibody comprising a capture moiety or different from the anti-PD-1 antibody comprising a capture moiety).

In another aspect, the disclosure provides methods for ex vivo T-cell expansion comprising the steps of:

(a) priming a sample of T cells expressing PD-1 isolated according to any of the methods disclosed herein, wherein the step of priming comprises:
(i) coating a culture plate on day −1 with priming factors;
(ii) seeding the population of T cells expressing PD-1 on day 0, wherein the step of seeding comprises:
   (1) adding a base media to the coated culture plate; and/or
   (2) adding an amount of isolated and/or enriched T cells to the base media to produce a seeding mixture in the coated culture plate;

(b) harvesting the primed T cells expressing PD-1;

(c) placing the harvested T cells expressing PD-1 in a seeding mixture and placing the seeding mixture into a non-treated culture plate;

(d) culturing the T cells expressing PD-1 in the seeding mixture;

(e) harvesting the T cells expressing PD-1 from the cultured seeding mixture;

(f) repeating steps (b)-(e) until a target number of expanded T cells is obtained.

In some embodiments, the priming factors used in the step of coating the culture plate include OKT3, soluble α-CD28, α-ICOS, α-ICOS (#140), α-LAG3, α-CD137, α-OX40, or any combination thereof. The term “priming factors,” as used herein, refers to additives used in the priming of the expansion culture. Such factors or additives are known in the art, and include, as non-limiting examples, IL-2, α-TIGIT, Iso, α-CD226, α-CD28, α-TIM3, α-LAG3, α-PD-1, Luperox, bezafibrate, or any combination thereof. In some embodiments, additives are added to the base media, and the additives may comprise IL-2, α-TIGIT, Iso, α-CD226, α-CD28, α-TIM3, α-LAG3, α-PD-1, α-OX40, Luperox, Bezafibrate, or any combination thereof.

In another aspect, the disclosure provides methods for treating a subject comprising administering to the subject a therapeutically effective amount of T cells expressing PD-1 isolated according to the methods disclosed herein. In some embodiments, the methods disclosed herein are used to treat a subject with cancer.

In one aspect, the disclosure provides methods for ex vivo T cell expansion, comprising:

(a) contacting a population of T cells with beads conjugated with anti-CD3 antibody, anti-ICOS antibody, or a combination thereof;

(b) incubating the bead and T cell mixture to expand the T cell population.

In another aspect, the disclosure provides methods for ex vivo T cell expansion wherein the T cell population comprises activated CD4 and CD8 cells.

In another aspect, the disclosure provides methods of treating a subject comprising administering to the subject a therapeutically effective amount of T cells expressing PD-1;
wherein the T cells expressing PD-1 are isolated by:
   (a) contacting the cell population with an amount of an anti-PD-1 antibody to produce an antibody-cell mixture, wherein the anti-PD-1 antibody comprises a capture moiety, and wherein the capture moiety is connected to the anti-PD-1 antibody via a linker;
   (b) contacting the antibody-cell mixture with an amount of magnetic beads, wherein the magnetic beads are capable of specifically binding the capture moiety on the anti-PD-1 antibody to produce a bead mixture;
   (c) passing the bead mixture through a magnetic field to isolate the magnetic beads and PD-1 expressing T cells bound thereto from the bead mixture; and
   (d) eluting the PD-1 expressing T cells from the magnetic field to isolate T cells expressing PD-1;
wherein a PD-1 expression level of the T cells expressing PD-1 isolated in step (d) is selected by adjusting one or more of:
   (i) the concentration of the anti-PD-1 antibody in the antibody-cell mixture;
   (ii) the length of the linker;
   (iii) the stoichiometric ratio of capture moiety to anti-PD-1 antibody (CAR);
   (iv) the ratio of anti-PD-1 antibody comprising a capture moiety to unmodified anti-PD-1 antibody;
   (v) the temperature at which either step (a) and/or step (b) is carried out;
   (vi) the concentration of T cells in the cell population;
   (vii) the concentration of T cells in the antibody-cell mixture and/or bead mixture;
   (viii) the flow rate at which the bead mixture is passed through the magnetic field;
   (ix) the length of time between the production of the antibody-cell mixture and step (b);
   (x) the length of time between production of the bead mixture and step (c); or
   (xi) the magnetic field strength; and

wherein the population of T cells expressing PD-1 is subjected to ex vivo expansion by:
(a) priming a sample of T cells expressing PD-1 isolated according to the any of the methods disclosed herein, wherein the step of priming comprises:
   (i) coating a culture plate on day −1 with priming factors;
   (ii) seeding the population of T cells expressing PD-1 on day 0, wherein the step of seeding comprises:
      (1) adding a base media to the coated culture plate; and/or
      (2) adding an amount of isolated and/or enriched T cells to the base media to produce a seeding mixture in the coated culture plate;
(b) harvesting the primed T cells expressing PD-1;
(c) placing the harvested T cells expressing PD-1 in a seeding mixture and placing the seeding mixture into a non-treated culture plate;
(d) culturing the T cells expressing PD-1 in the seeding mixture;
(e) harvesting the T cells expressing PD-1 from the cultured seeding mixture;
(f) repeating steps (b)-(e) until a target number of expanded T cells is obtained.

In some embodiments of any of the methods of expansion and/or treatment disclosed herein, an ICOS agonist is present during the priming step of the expansion method to drive T cell expansion. In some embodiments, the priming step corresponds to the first 4 days of stimulation. In some embodiments, IL-2 is dispensible for the first 4 days of expansion (i.e., during priming), but is required thereafter once ICOS agonism is removed.

Thus, in some embodiments, the priming factors used in T cell expansion include an ICOS agonist. In some embodiments, the ICOS agonist is an anti-ICOS antibody. In some embodiments, T cells are primed for 4 days. In some embodiments, culturing of T cells after priming is conducted in the presence of IL-2.

In further embodiments, the priming factors used in T cell expansion include an OX40 agonist. In some embodiments, the OX40 agonist is an anti-OX40 antibody. In some embodiments, the anti-OX40 antibody is MEDI0562. Disclosure related to MEDI0562 can be found in U.S. Pat. No. 9,738,723, incorporated here by reference in its entirety. In some embodiments, T cells are primed for 4 days. In some embodiments, culturing of T cells after priming is conducted in the presence of IL-2.

In another aspect, the disclosure provides methods of treating cancer in a subject, including first moblilzing PD-1⁺ tumor-infiltrating lymphocytes (TIL). In certain aspects, this moblilzation is mediated by administration of Plerixafor, IL-10, Vemurafenib, CXCL2 or tremelimumab. In one specific aspect, the methods comprising: (a) administering an anti-CTLA-4 antibody to the subject in an amount effective to mobilize PD-1⁺ tumor-infiltrating lymphocytes (TIL) into the peripheral blood; (b) harvesting PD-1⁺ TIL from the peripheral blood of the subject; (c) expanding the harvested PD-1⁺ TIL; and (d) administering the expanded PD-1⁺ TIL to the subject. In some embodiments, the harvested PD-1⁺ TIL are expanded according to any of the methods of expansion disclosed herein. In some embodiments, the anti-CTLA-4 antibody is tremelimumab. Disclosure related to tremelimumab can be found in U.S. Pat. No. 6,682,736, incorporated here by reference in its entirety.

In one aspect, the methods disclosed herein further include measurement of tumor mutational burden (TMB). These measurements can be taken before, during or after treatment. On certain aspects, the methods disclosed herein may be more successful for patients with high TMB. In one aspect, TMB is determined by measuring level of circulating tumor DNA (ctDNA). Under normal conditions, cell-free DNAs (cfDNA) are observable in the blood only in low amounts, however they are not efficiently cleared in response to exhaustive exercise, inflammation, or occurrence of disease. For example, circulating DNAs deriving from cancer cells represents a distinct and measurable component of the cfDNA in cancer patients. This ctDNA fraction of cfDNA can be useful for classifying tumors and cancer disease, such as stratifying cancer patients, allowing for administration of therapies that are more likely to be effective, as well as for modification of current therapies that are less likely to provide clinical improvement. Methods for use and measurement of ctDNA can be found e.g., in US Publication No. US 2018/0282417.

### EXAMPLES

The Examples that follow are illustrative of specific embodiments of the disclosure, and various uses thereof. They are set forth for explanatory purposes only, and should not be construed as limiting the scope of the disclosure in any way.

### Example 1. Enrichment of PD-1 Expressing T Cells

Donor blood was obtained in the form of human peripheral blood through standard leukapheresis or blood draw protocols. The red blood cells were removed from the donor blood by centrifugation protocols known in the art. Briefly, the peripheral blood was centrifuged, the supernatant discarded, the pellet was resuspended in ammonium chloride potassium (ACK) buffer, the ACK pellet suspension was incubated at room temperature, and the cells were washed twice in wash buffer to produce a cell pellet.

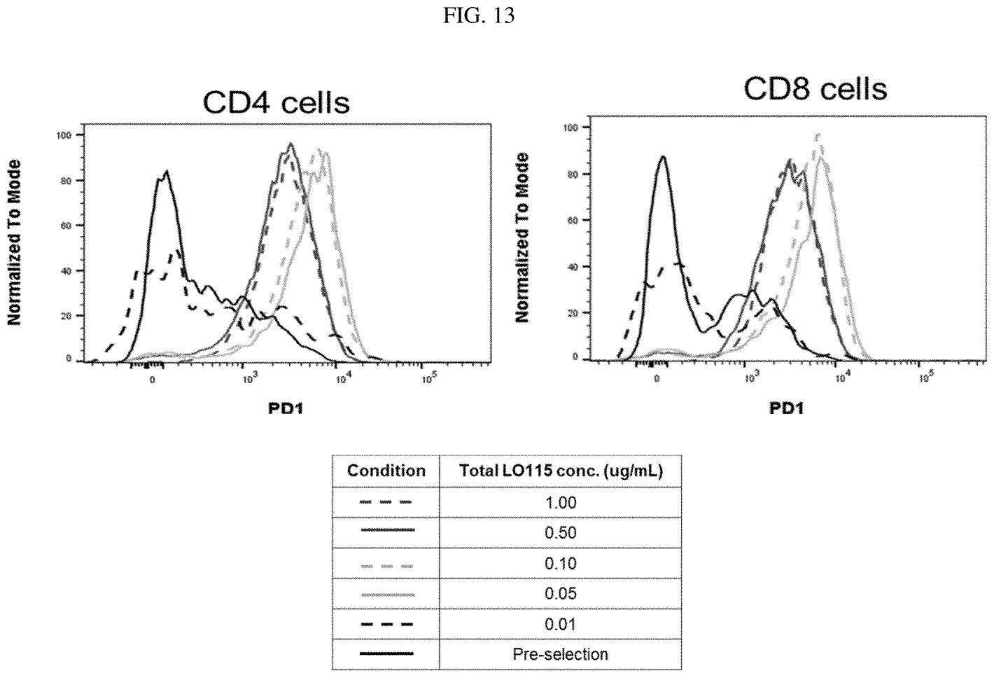

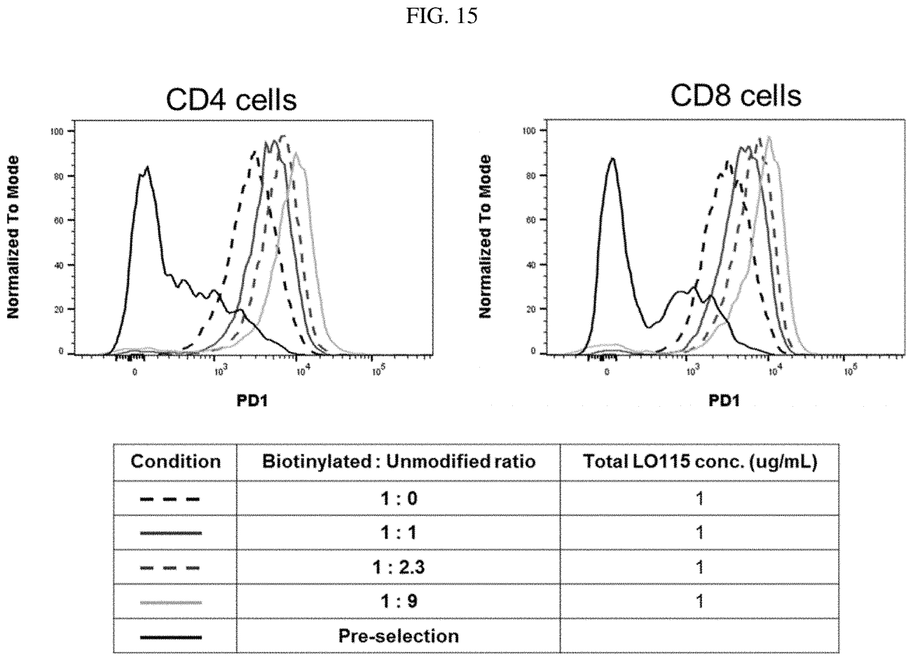

Enrichment of the PD-1 expressing cells was then performed. The washed cell pellet was resuspended in wash buffer and cell count determined. Wash buffer was added to bring the cell density to 100×10⁶ cells per mL. 1 μg/mL MEDI3097 antibody was added to the cells to produce an antibody-cell mixture comprising cells and MEDI3097 antibody. The antibody mixture was incubated at 4° C. for 15 minutes and then centrifuged and washed twice with wash buffer. Wash buffer was added to bring cell density to 125×10⁶ cells per mL. Anti-biotin beads were then added to the cells at a concentration of 2 mL beads per 1 billion cells to produce a bead mixture. The bead mixture was incubated at 4° C. for 15 minutes, and then was centrifuged, washed once, and the cells resuspended in wash buffer at a concentration of 200×10⁶ cells/mL total cells. The resuspended bead mixture was then loaded onto a magnetic column. The column was washed three times with wash buffer. T cells with the varying PD-1 expression levels were eluted. Additionally, LO115 anti-PD-1 antibody was tested and found to produce similar results to MEDI3097 (see FIGS. 13 and 15).

### Example 2. Tunable Enrichment of PD-1 Expressing T Cells

Enrichment of PD-1 expressing T cells was performed after red blood cells were removed from leukapheresis products. The washed cell pellet was resuspended in wash buffer and cell count determined. Wash buffer was added to bring cell the density to 100×10⁶ cells per mL. 1 μg/mL MEDI3097 antibody was added to the cells to produce an antibody-cell mixture comprising cells and MEDI3097 antibody. A number of process variables were identified as having a significant impact on the selection of PD-1 high or low expressing T cells.

In one experiment, the effect of biotin to anti-PD-1 antibody ratio (BAR) on PD-1 expression level of enriched cells was assessed. As shown in FIG. 5B, a low BAR provided high PD-1 expression level and a low yield, while a high BAR provided low PD-1 expression level and high yield. The effect of BAR adjustment plateaued around 3.5-4.0 for MEDI0680 at 4° C.

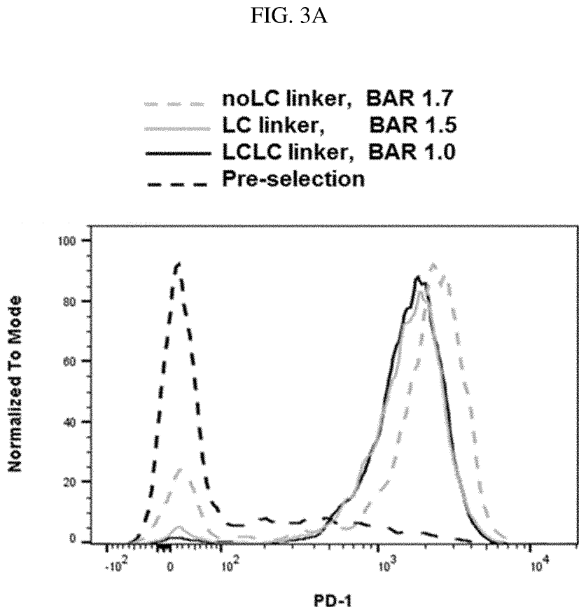

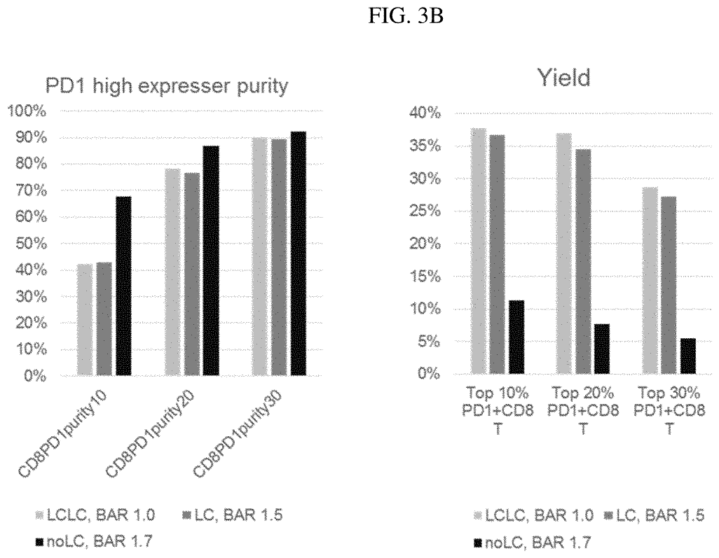

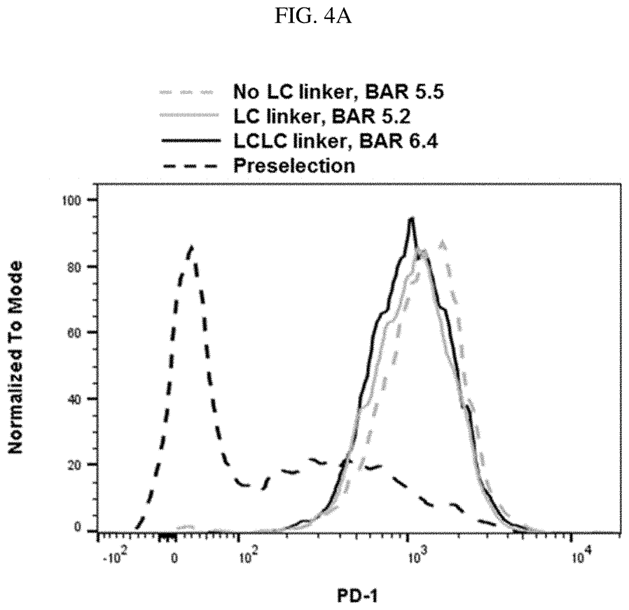

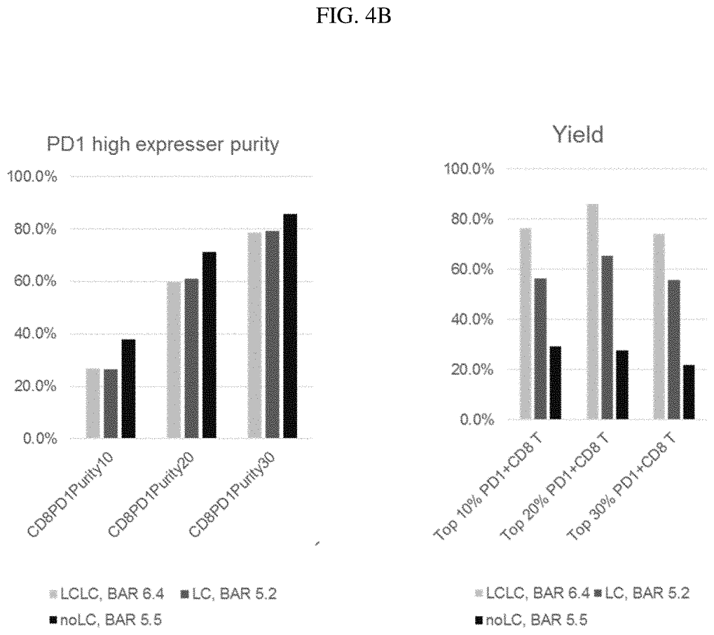

In another experiment, the impact of the length of the linker between the capture moiety and the anti-PD-1 antibody was investigated. Linkers having a length between about 1 Å and about 50 Å were tested. The impact of linker length on PD-1 high expressers was also affected by BAR. FIG. 3 shows the PD-1 expression, as well as PD-1 high expresser purity and yield at low BAR and various linker lengths, while FIG. 4 shows the selection results at high BAR and various linker lengths.

Additionally, the concentration of anti-PD-1 antibody was found to impact the selection of PD-1 high or PD-1 low expressing T cells. A low concentration of anti-PD-1 antibody resulted in high PD-1 expression level and low yield. In contrast, high anti-PD-1 antibody concentration resulted in low PD-1 expression level and high yield. The impact of anti-PD-1 antibody concentration on PD-1 expression level plateaued around 5.0 μg/mL for MEDI0680 at 4° C. Initial total cell concentration did not affect selection outcome, as shown in FIG. 5.

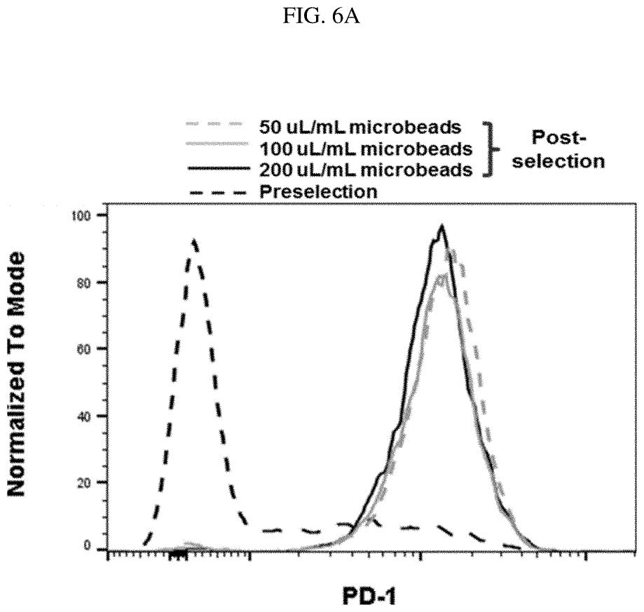

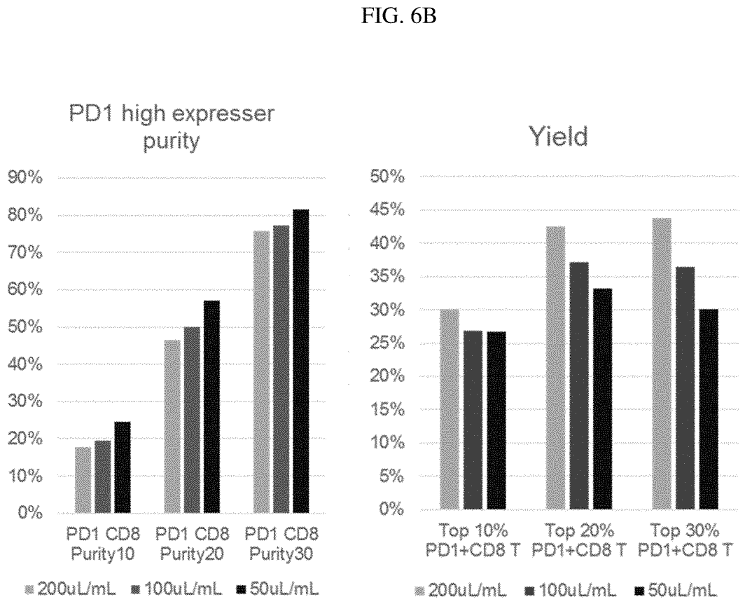

Furthermore, the concentration of anti-biotin microbeads was found to impact the selection of PD-1 expression levels of T cells, as shown in FIG. 6A. PD-1 expression level was found to negatively correlate with bead concentration: as the amount of microbeads that were added was increased from 50 μL to 100 μL to 200 μL of microbead suspension per mL of cell suspension, the percentage of cells at each CD8PD-1 purity level (10%, 20%, and 30%) decreased (FIG. 6B, left). The yield of T cells expressing high levels of PD-1 was found to positively correlate with microbead concentration: as the amount of microbeads that were added was increased from 50 μL to 100 μL to 200 μL of microbead suspension per mL of cell suspension, the yield of cells at each CD8PD-1 purity level (10, 20, and 30) increased.

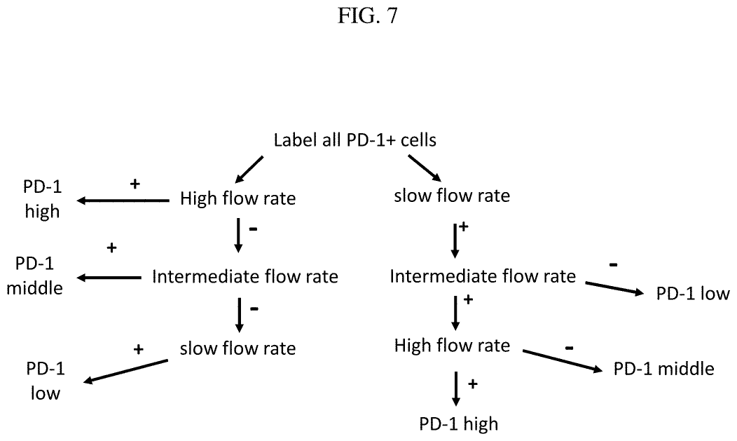

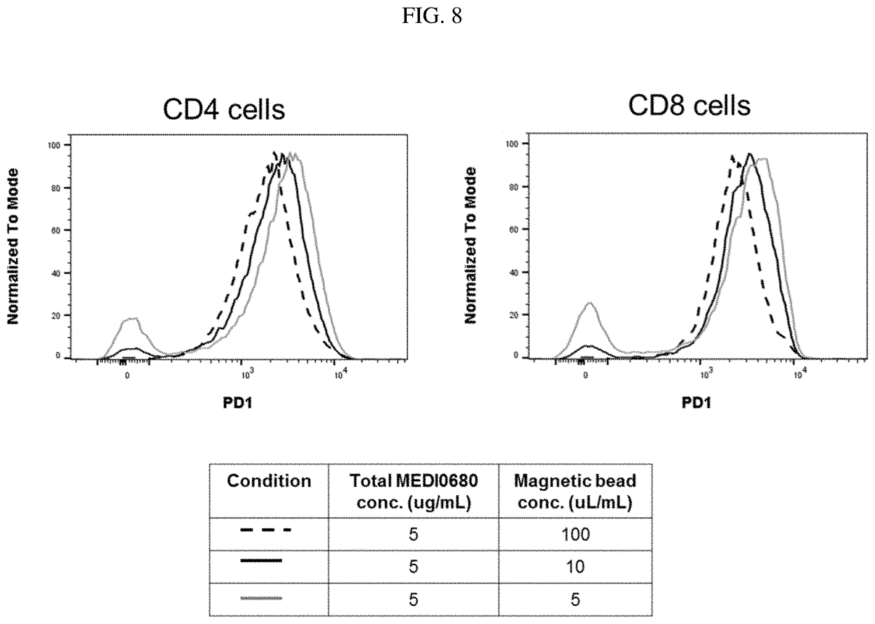

The effect of the flow rate at which the resuspended bead mixture was passed through the magnetic column was also investigated (FIG. 7, left branch). When the bead mixture was first passed through the column at a high flow rate, PD-1 high expressing T cells were selected and a negative fraction of the bead mixture remained. The negative fraction of the bead mixture was passed through the column a second time at an intermediate flow rate. The intermediate flow rate selected PD-1 intermediate (“PD-1 middle”) expressing T cells and left a negative fraction of the bead mixture. The negative fraction of the bead mixture was passed through the column a third time, at a lower flow rate. This lower flow rate selected PD-1 low expresser T cells.

When the bead mixture was first passed through the column at a low flow rate (FIG. 7, right branch), the captured cells were eluted and passed through the column a second time at an intermediate flow rate. The intermediate flow rate produced captured cells and a negative fraction containing PD-1 low expresser T cells. The captured cells were again eluted and then were passed through the column a third time at a high flow rate, which produced captured cells and a negative fraction containing PD-1 intermediate (“PD-1 middle”) expresser T cells. The captured fraction contained PD-1 high expresser T cells, which were eluted from the column.

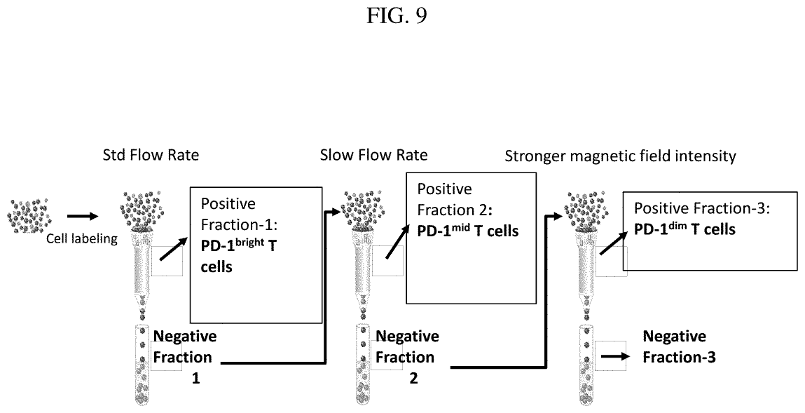

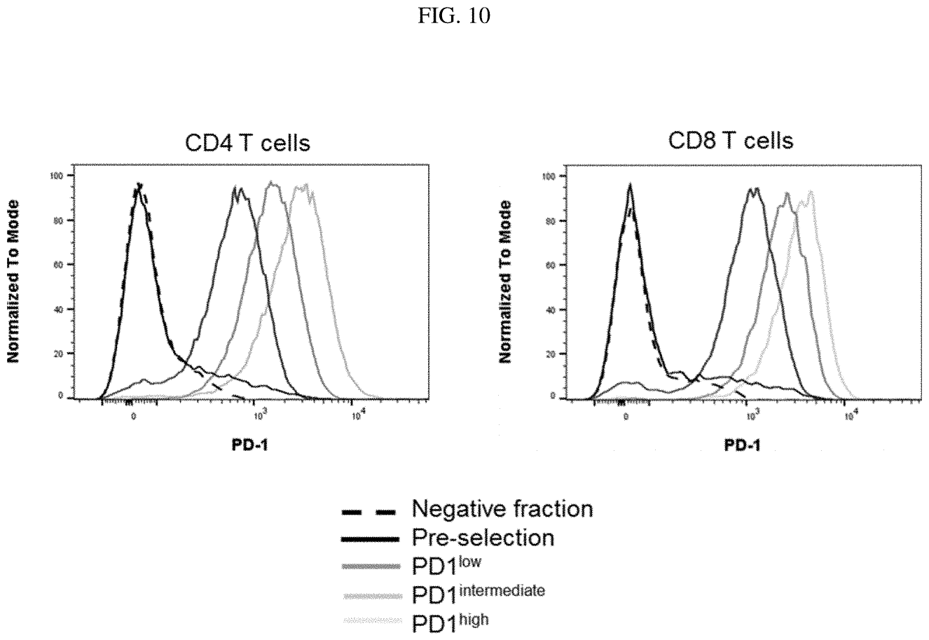

As shown in FIG. 9, combinations of various flow rate and magnetic field intensity were also tested. PD-1 positive cells were first passed through the column at a high flow rate, PD-1 high expressing T cells were selected, and a negative fraction of the bead mixture remained. The negative fraction of the bead mixture was passed through the column a second time at an intermediate flow rate. The intermediate flow rate selected PD-1 intermediate expressing T cells and left a second negative fraction of the bead mixture. The second negative fraction was passed through the magnetic field a third time, at a stronger magnetic field intensity, selecting PD-1 low expressing cells. FIG. 10 shows the selection results for both CD4+ T cells and CD8+ T cells, where the first flow was performed at a high flow rate, followed by low flow rate, and finishing with a stronger magnetic field intensity.

### Example 3. Formulaic Method for Tunable Enrichment of PD-1 Expressing T Cells

The enrichment of T cells with distinct PD-1 expression levels was determined to follow a mathematical model consistent with the experiments described herein, as set forth in Formula 1:

$$N ( biotin ) = P ( biotin ) \times N = \frac{N \times α \times [ Ab ] i}{{K}_{d} + [ Ab ] i}$$

wherein:

N(biotin) is the number of expected accessible biotin molecules on each PD-1+ cell, negatively correlating with PD-1 expression level of isolated cells;

N is the number of PD-1 antigen-binding sites on the cells;

[Ab]ᵢ is the total concentration of the anti-PD-1 antibody;

α is the ratio of anti-PD-1 antibody with accessible capture moiety to the total anti-PD-1 antibody; and

K_{d} is the dissociation constant of the anti-PD-1 antibody at the incubation temperature, i.e., the ratio of K_{off}/Kₒₙ where, Kₒₙ×[A_{b}]×[A_{g}]=K_{off}×[A_{b}A_{g}] at equilibrium state.

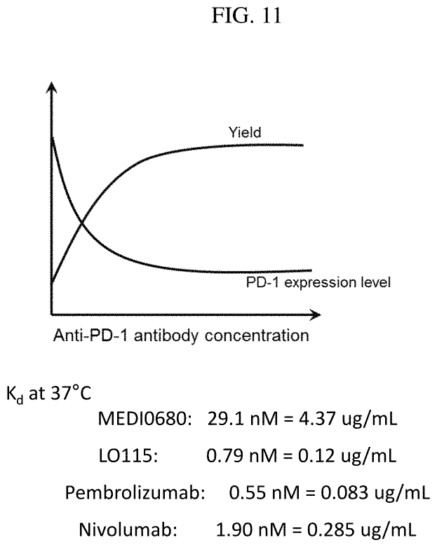

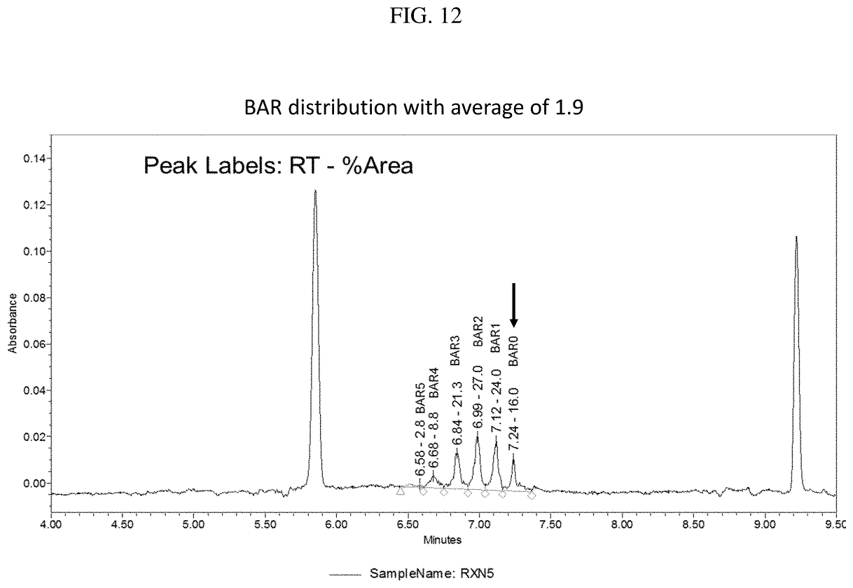

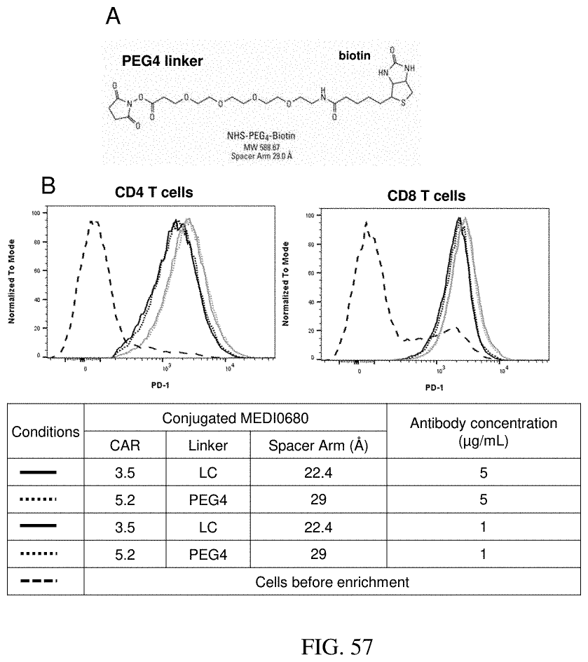

In the mathematical model, the concentration of the anti-PD-1 antibody is critical to the selection process. As seen in Example 2 above, Formula 1 is consistent with experimental results. The [Ab]ᵢ has a significant effect if it is close to K_{d} (see FIG. 11, showing the concentration of anti-PD-1 antibody with the K_{d} values). When [Ab]ᵢ is >>>K_{d}, the effect of the concentration of the anti-PD-1 antibody on PD-1 expressing cell isolation (overall yield and PD-1 expression level of isolated cells) is plateaued. The experimental results in FIG. 5 are consistent with the model's prediction. When BAR is low, there remains unmodified anti-PD-1 antibody (see FIG. 12, black arrow) and then a is <1. As BAR increases, the a ratio of biotinylated MEDI0680 increases to 1 and the effect of BAR on PD-1 expressing cell isolation (overall yield and PD-1 expression level of isolated cells) first changes and then plateaus. The experimental results in FIG. 5 are consistent with the model's prediction. With a short linker, such as one with 13.5 Å length (see FIG. 2), the capture moiety biotin may not be accessible to anti-biotin beads on some primary amine sites, and thus the a ratio is <1 for these biotinylated MEDI0680 molecules. When conjugated with long linkers, such as those over 17 Å, all capture moiety biotin can be accessible to anti-biotin beads and thus the a ratio is 1 for these biotinylated MEDI0680 molecules. As a result, biotinylated MEDI0680 with a short linker can enrich T cells with higher PD-1 expression level than those with long linker at similar BAR level. This prediction is confirmed in experimental results (see FIGS. 3, 4, and 5). This prediction also holds true for different types of linkers (e.g., PEG4 with 29 Å, see FIG. 2) and experimental results showed biotinylated MEDI0680 using PEG4 linker isolated T cells with a similar PD-1 expression level to that for T cells isolated using biotinylated MEDI0680 linked by LC linker with 22.4 Å length when antibody concentration is the same (see FIG. 57).

When applying Formula 1, above, where the number of antibody molecules is much larger than the number of cells expressing PD-1, which should be true when the concentration is 1 μg/mL, the probability for one PD-1 molecule having at least one accessible biotin molecule is determined using Formula 2:

$$P ( biotin ) = P ( Ab ) * α = \frac{α * {[ Ab ]}_{i}}{{K}_{d} + {[ Ab ]}_{i}}$$

The probability for one PD-1 expressing molecule to have one antibody is based on the equilibrium state provided in the equation of Formula 3 below:

$$P ( Ab ) = \frac{[ Ab Ag ]}{{[ Ag ]}_{i}} = \frac{{[ Ab ]}_{i}}{{K}_{d} + {[ Ab ]}_{i}} ,$$

wherein the initial anti-PD-1 antibody concentration is [A]ᵢ. The variable α may be adjusted by changing: the biotin to anti-PD-1 antibody ratio, the linker length between the capture moiety and anti-PD-1 antibody, or the ratio of anti-PD-1 antibody with capture moiety and unmodified anti-PD-1 antibody, which can be the same as the anti-PD-1 antibody with capture moiety or different from the anti-PD-1 antibody with capture moiety, or any combination of these factors. This is true for any anti-PD-1 antibody, including MEDI0680 with low binding affinity and LO115 with high binding affinity.

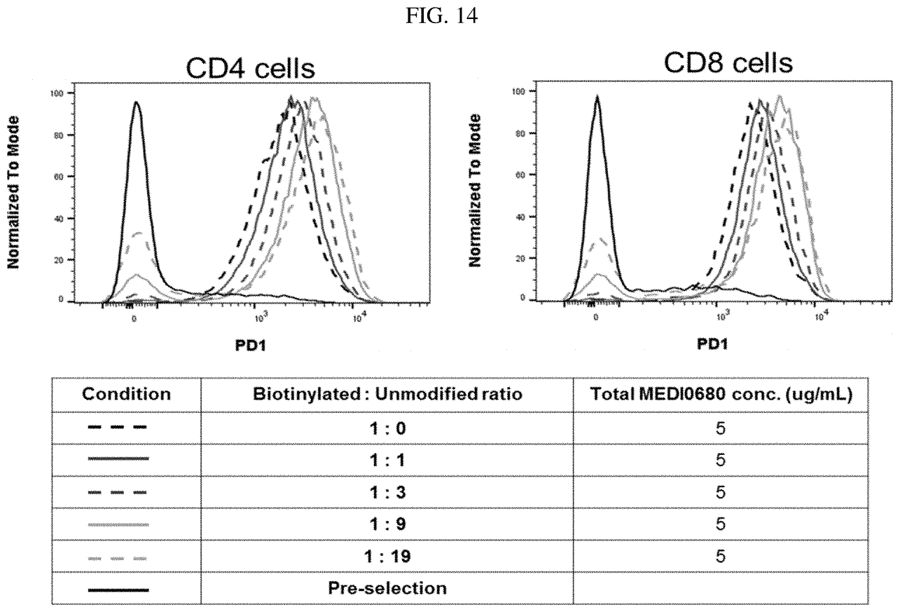

FIG. 13 shows that when the biotinylated PD-1 antibody (LO115) concentration is adjusted, the expression of PD-1 high T cells increases as the concentration of the biotinylated antibody decreases. FIG. 14 shows that when the ratio of anti-PD-1 with capture moiety (biotinylated) and unmodified anti-PD-1 antibody is adjusted for MEDI0680, the PD-1 expression level of isolated cells increased as the ratio of unmodified anti-PD-1 antibody increased. This is observed even when the total anti-PD-1 antibody concentration is constant. FIG. 15 shows similar results for LO115.

### Example 4. Rapid Expansion of PD-1 Expressing T Cells

T cells enriched from leukapheresis products as described in Examples 1 and 2 were expanded as follows. Base media was prepared by using X-Vivo 15 media with 1× L-glutamine, 10 mM HEPES, 5% CTS Immune Cell SR, and 500 U/mL IL-2. Priming medium was prepared by adding 1 μg/mL α-CD28 to the prepared base media. In some experiments, expansion medium was prepared by adding 0.01 μM Luperox to prepared base media.

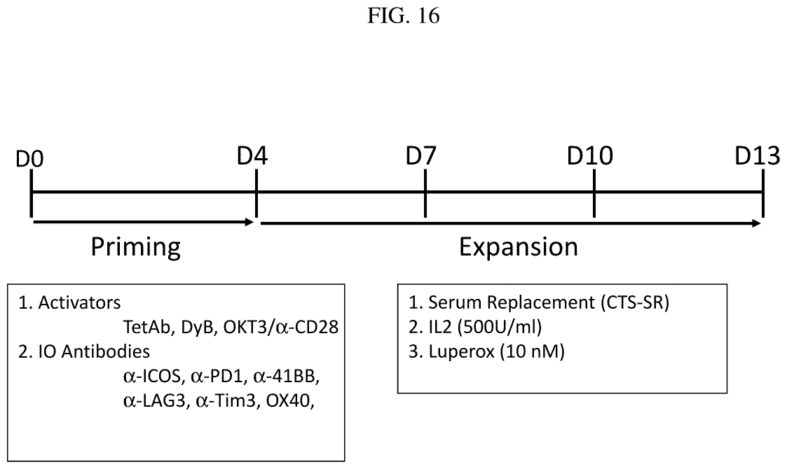

A day before the enriched T cells were to be seeded for expansion, a non-treated 24-well culture plate was primed by coating the plate overnight with the following priming factors: 1 μg/mL OKT3, a monoclonal antibody to human T cells, and varying concentrations of α-ICOS (from 0 to 5 μg/mL) in 500 μL phosphate buffered saline in each well. A schematic of the priming is shown in FIG. 16. The priming factors were incubated overnight at 4° C.

The next day (day 0), the primed 24-well plate was washed twice with PBS, and PD-1 expressing cells were enriched using the enrichment protocols described herein. The enriched PD-1 cells were suspended in priming medium at 0.5×10⁶ cell/mL. The suspended cells were then seeded on the primed plate, and then were incubated at 37° C.

On day 4, the seeded cells were harvested from the 24-well plate, counted, resuspended, and then 1.5 to 2.5×10⁶ cells were seeded in expansion medium on a 6-well plate, each well containing a 10 cm² gas permeable membrane and 40 mL media capacity (Wilson Wolf, G-Rex6 Well Plate—P/N 80240M).

On day 7, the cells were harvested, counted, resuspended, and then 5×10⁶ cells were plated in each well of a 6-well plate in expansion medium. This process was repeated on day 10.

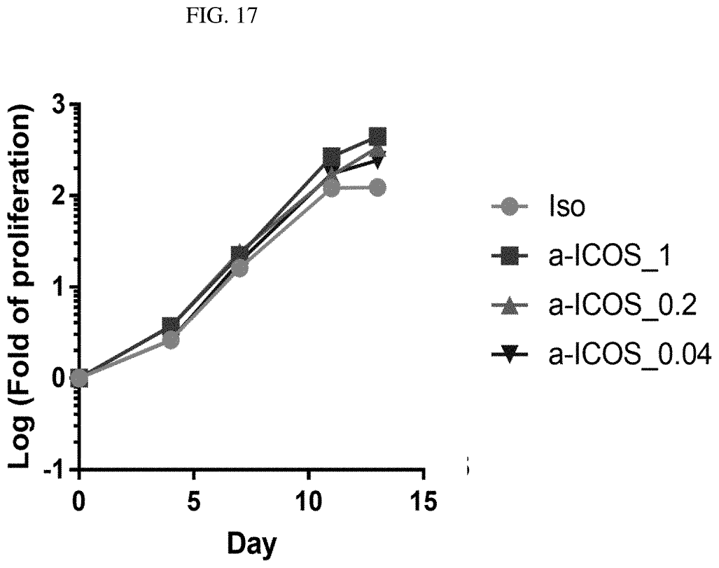

On day 13, the cells were harvested, counted, and the expansion concluded. FIG. 17 shows the cell proliferation of the PD-1 expressing cells for varying concentrations (0, 0.04, 0.2, and 1 μg/mL) of α-ICOS.

### Example 5. Comparison of Rapid Expansion Manual Priming and TetAb Priming of PD-1 Expressing T Cells

T cells enriched from leukapheresis products as described in Examples 1 and 2 were expanded as described in Example 4. Concentrations of 1 μg/mL of α-ICOS were used in priming the 24-well plate. Briefly, PD-1 expressing cells enriched from leukapheresis products were suspended in priming medium at 0.5×10⁶ cell/mL. The suspended cells were then seeded on the primed plate and α-CD28 or TetAb (no additional α-CD28) were added in soluble. The cells were then incubated at 37° C.

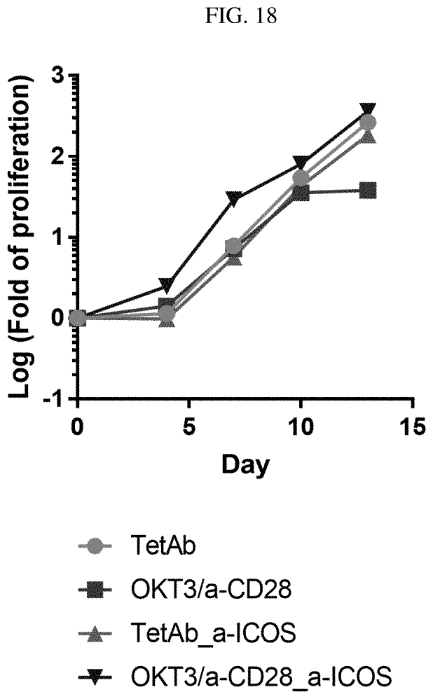

On day 13, the cells were harvested, counted, and the expansion concluded. FIG. 18 shows the comparison results of T cell proliferation, where the manual priming showed greater efficacy for rapid expansion.

### Example 6. Proliferation Efficacy of T Cell Expansion with α-ICOS Clones

As a comparison of proliferation efficacy of PD-1 expressing T cell expansion, varying concentrations of α-ICOS or varying concentrations of different α-ICOS clones were used in the expansion protocol described in Example 4. Concentrations of 0.2-5 μg/mL of α-ICOS and the α-ICOS clones #138 and #140 were used in priming the 24-well plate.

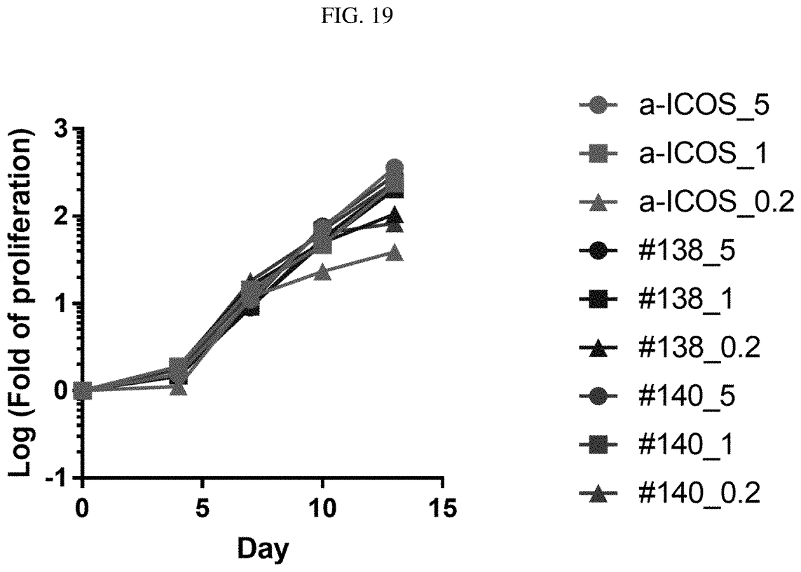

On day 13, the cells were harvested, counted, and the expansion concluded. FIG. 19 shows the cell proliferation of the PD-1 expressing cells at varying concentrations of α-ICOS and the α-ICOS clones where α-ICOS and α-ICOS clone #140 show similar proliferation efficiency.

### Example 7. Varying Duration of Chemical Treatment in T Cell Expansion

T cells enriched from leukapheresis products as described in Examples 1 and 2 were expanded as described in Example 4. Concentrations of 1 μg/mL of α-ICOS were used in priming the 24-well plate. 2 μM Bezafibrate or 0.05 μM Luperox was added to the cultures at varying starting points and for varying lengths of time. The variations were matched between the chemicals, where Bezafibrate or Luperox were added to the culture media at: 1) day 0 until day 4; 2) day 0 until day 7; 3) day 0 until day 10; 4) day 0 until day 13; 5) day 4 until day 13; 6) day 7 until day 13; and 7) day 10 until day 13.

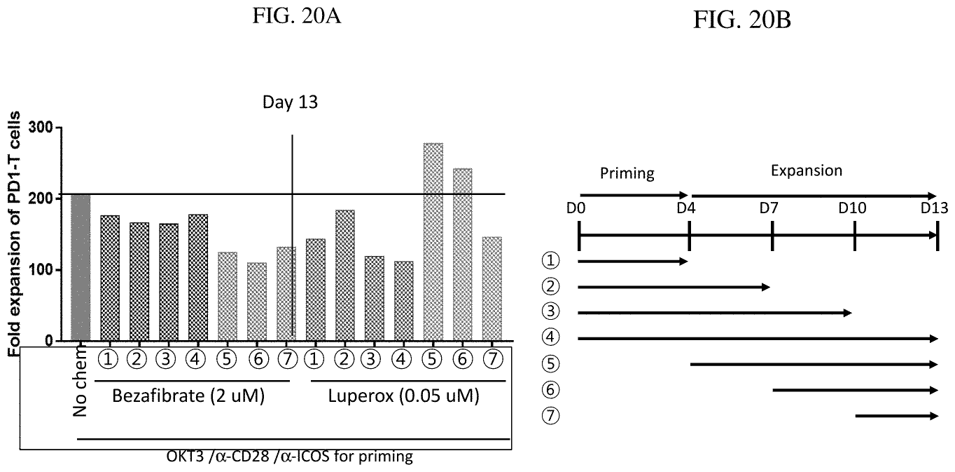

On day 13, the cells were harvested, counted, and the expansion ended. FIG. 20 shows the fold expansion of the PD-1 expressing cells at varying starting points and durations of chemical treatment.

### Example 8. Varying Duration and Concentration of Chemical Treatment in T Cell Expansion

T cells enriched from leukapheresis products as described in Examples 1 and 2 were expanded as described in Example 4. Concentrations of 1 μg/mL of α-ICOS were used in priming the 24-well plate. Briefly, PD-1 expressing cells enriched from leukapheresis products were suspended at 0.5×10⁶ cell/mL in priming medium with or without 2 μM Bezafibrate. The suspended cells were then seeded on the primed plate and incubated at 37° C.

On day 4, 1.5 to 2.5×10⁶ cells in expansion medium with or without 2 μM Bezafibrate or varying concentrations of Luperox between 0.01 to 1.25 μM were reseeded on a G-Rex 6 Well Plate.

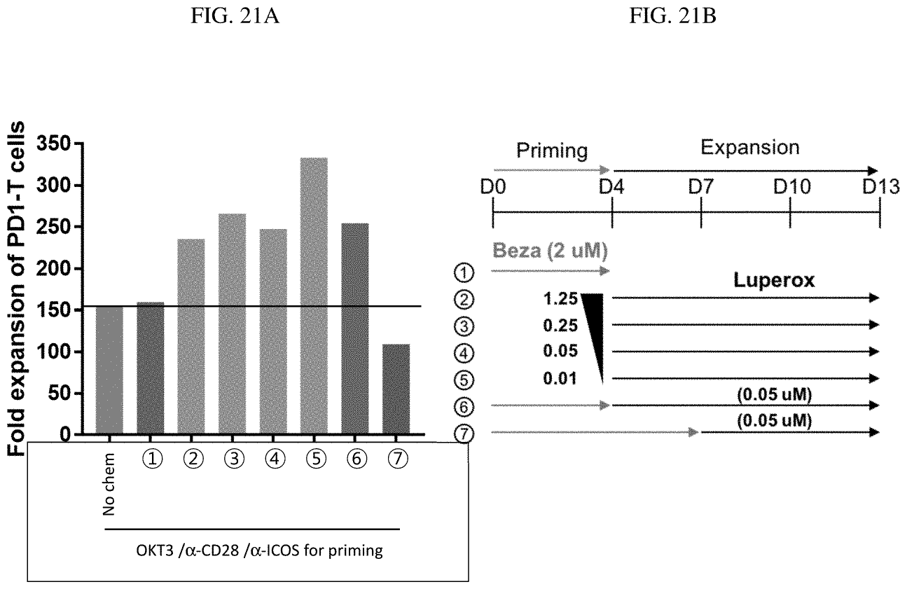

On day 13, the cells were harvested, counted, and the expansion ended. FIG. 21 shows the fold expansion of PD-1 expressing cells for varying chemical treatments and concentrations.

### Example 9. 6-Well Optimal Reseeding Rapid Expansion of PD-1 Expressing T Cells

T cells enriched from leukapheresis products as described in Examples 1 and 2 were expanded as described in Example 4. Concentrations of 1 μg/mL of α-ICOS or an α-ICOS clone (#140) were used in priming the 24-well plate.

On day 4, 7 and 10, 5×10⁶ cells were re-seeded in base medium with or without 0.01 or 0.05 μM Luperox on a G-Rex6 Well Plate.

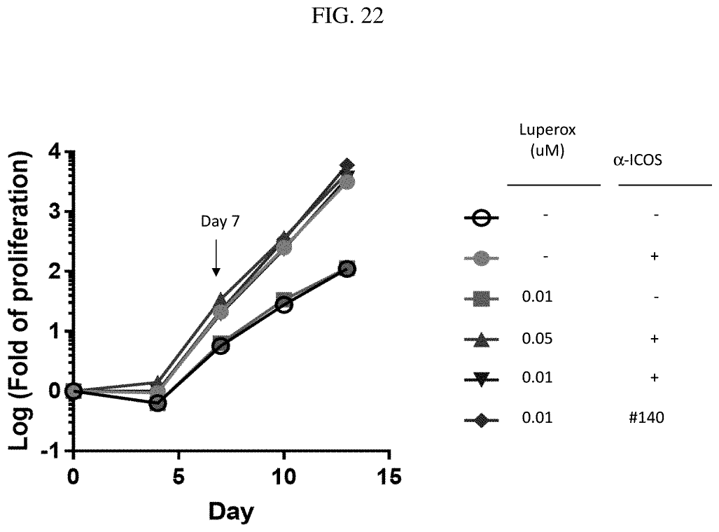

On day 13, the cells were harvested, counted, and the expansion ended. FIG. 22 shows the fold expansion of PD-1 expressing cells for the 6-well optimal reseeding process.

### Example 10. 6-Well Multi-Disciplinary Rapid Expansion of PD-1 Expressing T Cells

T cells enriched from leukapheresis products as described in Examples 1 and 2 were expanded as described in Example 4. Concentrations of 1 μg/mL of α-ICOS clone (#140) were used in priming the 24-well plate.

On day 4, 7 and 12, the 5×10⁶ cells were re-seeded in base medium with or without 0.01 μM Luperox on a G-Rex6 Well Plate.

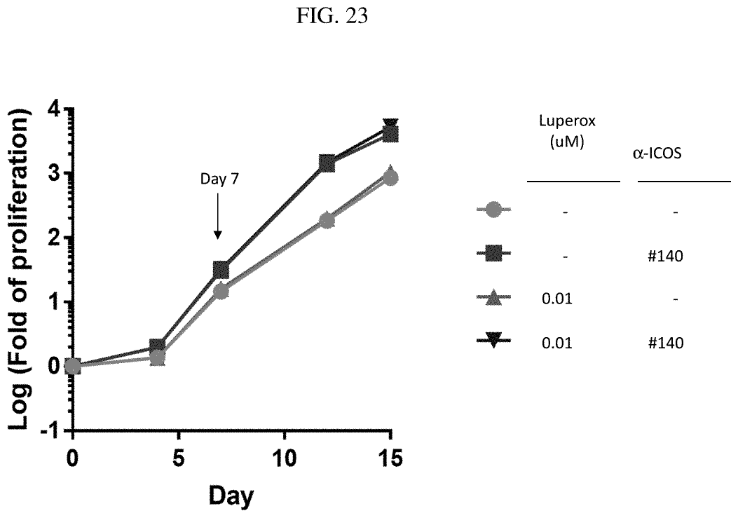

On day 15, cells were harvested, counted, and the expansion ended. FIG. 23 shows the fold expansion of the PD-1 expressing cells for the multi-disciplinary rapid expansion process.

### Example 11. Full Scale Expansion of PD-1 Expressing T Cells

T cells enriched from leukapheresis products as described in Examples 1 and 2 were expanded as described in Example 4 through a G-Rex 100M vessel full scale. Concentrations of 1 μg/mL of α-ICOS or an α-ICOS clone (#140) were used in priming the 24-well plate. On day 4, the 1.5 to 2.5×10⁶ cells were seeded in expansion medium with or without 0.01 μM Luperox on a G-Rex6 Well Plate.

On day 7, the cells were harvested and counted. Whole cells were placed in a 1000 mL media capacity vessel with a 100 cm² gas permeable membrane (Wilson Wolf, G-Rex100M-P/N 81100S) in 500 mL base medium with or without 0.01 μM Luperox. On day 10 of the expansion, the cell suspension was counted and 300 mL base medium with or without 0.01 μM Luperox was added to the vessel.

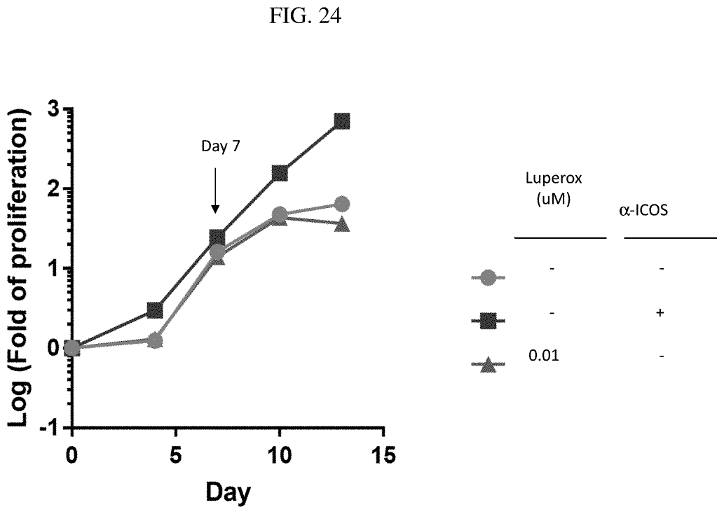

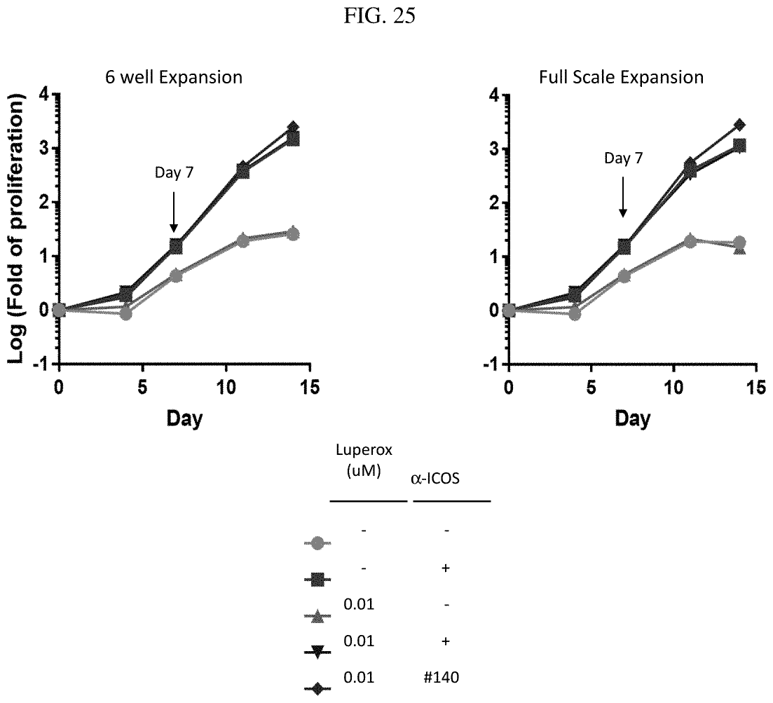

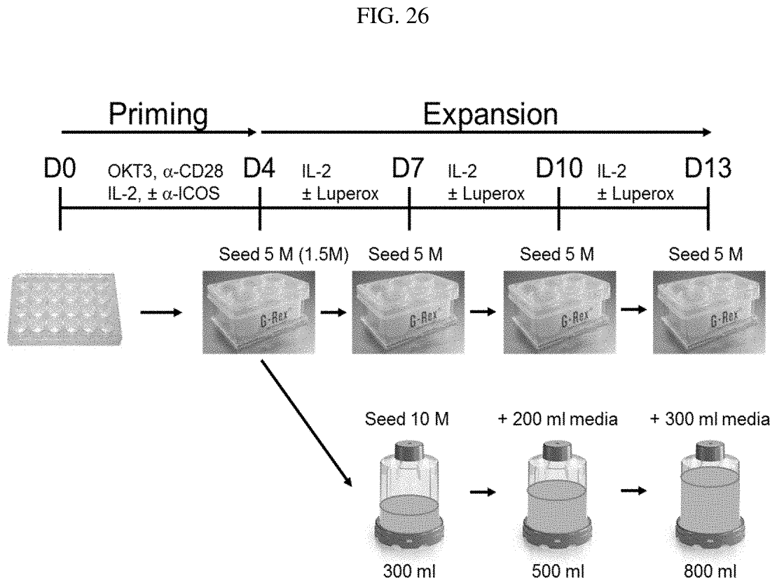

On day 13, the cells were harvested, counted, and the expansion ended. FIG. 24 shows the fold proliferation of PD-1 expressing cells. FIG. 25 shows a comparison of expansion using the optimal reseeding process and the 100 million full scale culture process, while FIG. 26 shows the scheme used for optimal reseeding culture and full scale culture.

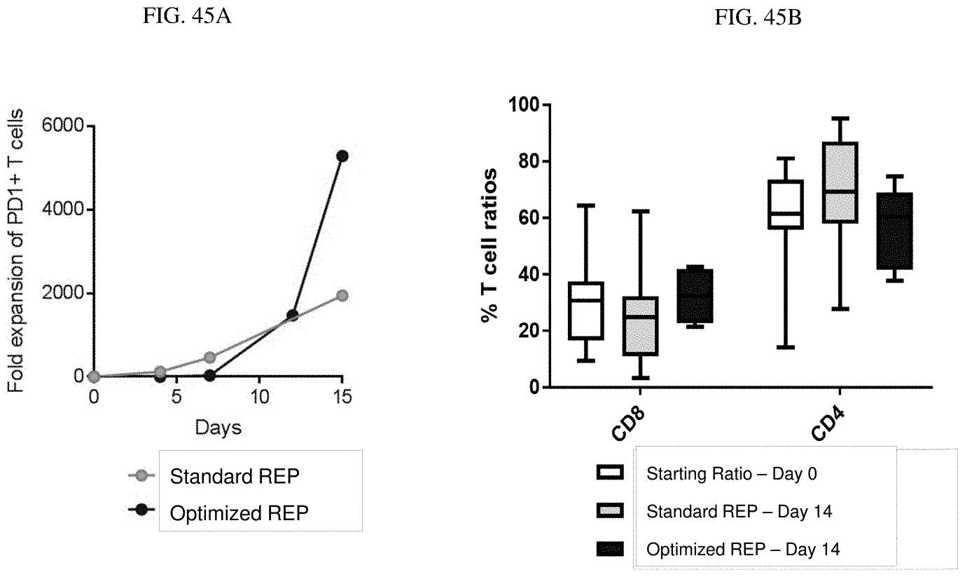

FIGS. 45A and 45B show the advantageous fold expansion of PD-1⁺ T cells (FIG. 45A) and percent CD4/CD8 T cell ratios (FIG. 45B) resulting from the improved rapid expansion protocol disclosed herein as compared to a standard rapid expansion protocol (as described in Dudley et al., 2003, J. Immunother. 26(4): 332-42). These results demonstrated that the rapid expansion protocol disclosed herein drives PD-1⁺ T cell expansion and maintains CD4/CD8 T cell ratios more favorably when compared to the standard rapid expansion protocol.

### Example 12. Antibody Testing on PD-1 Expressing T Cells Expansion

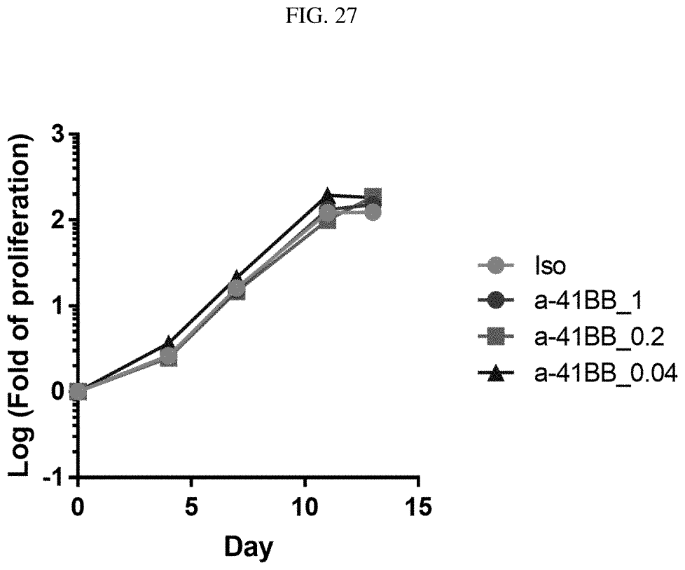

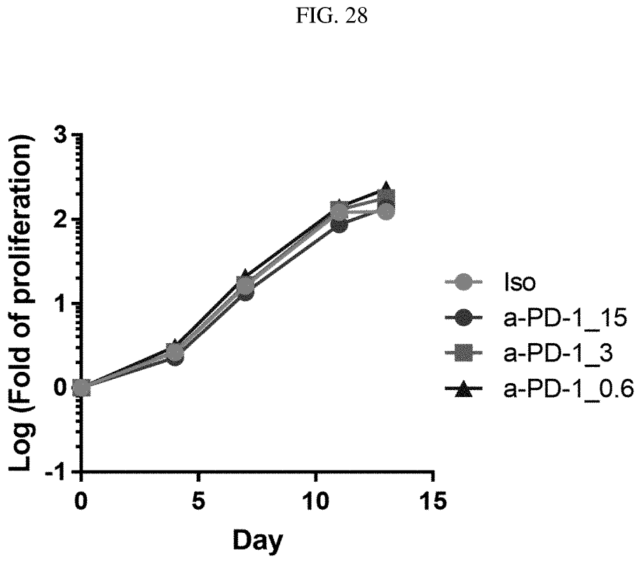

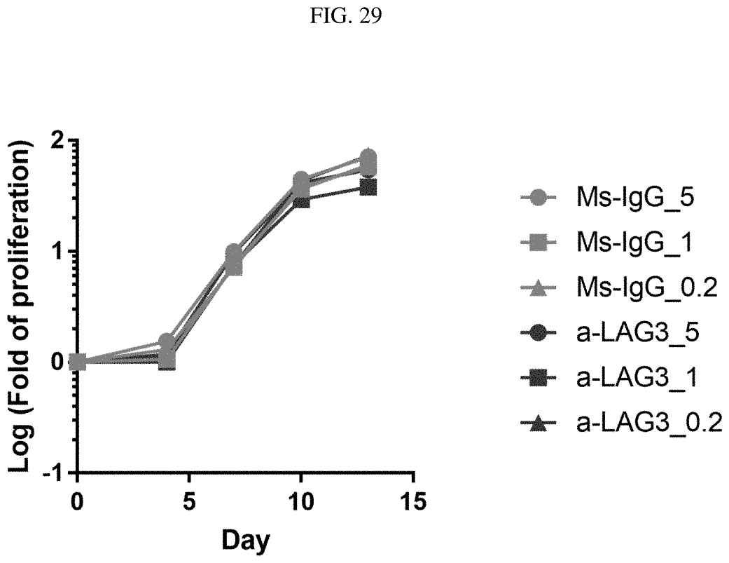

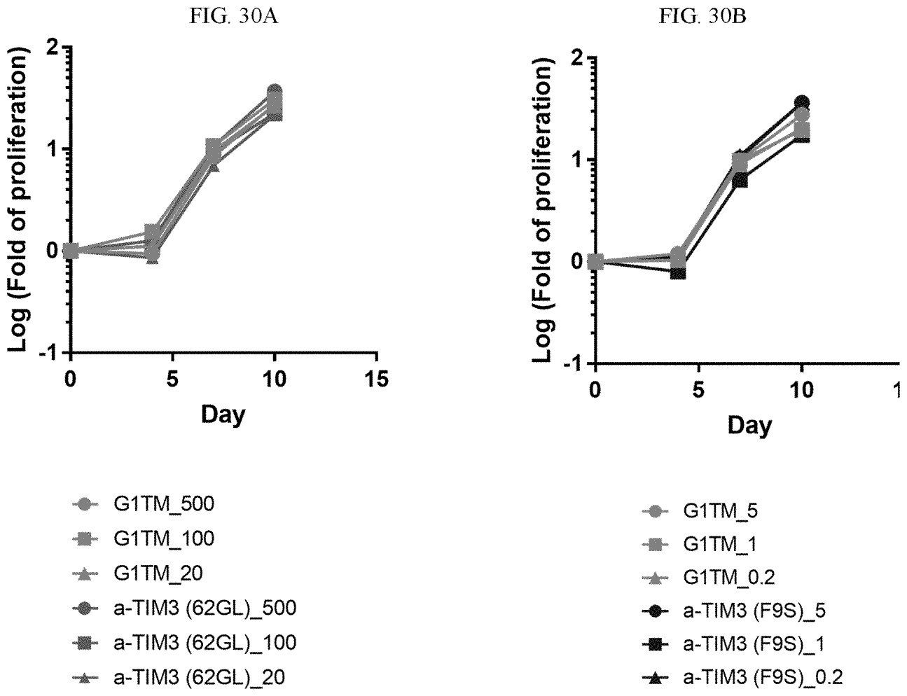

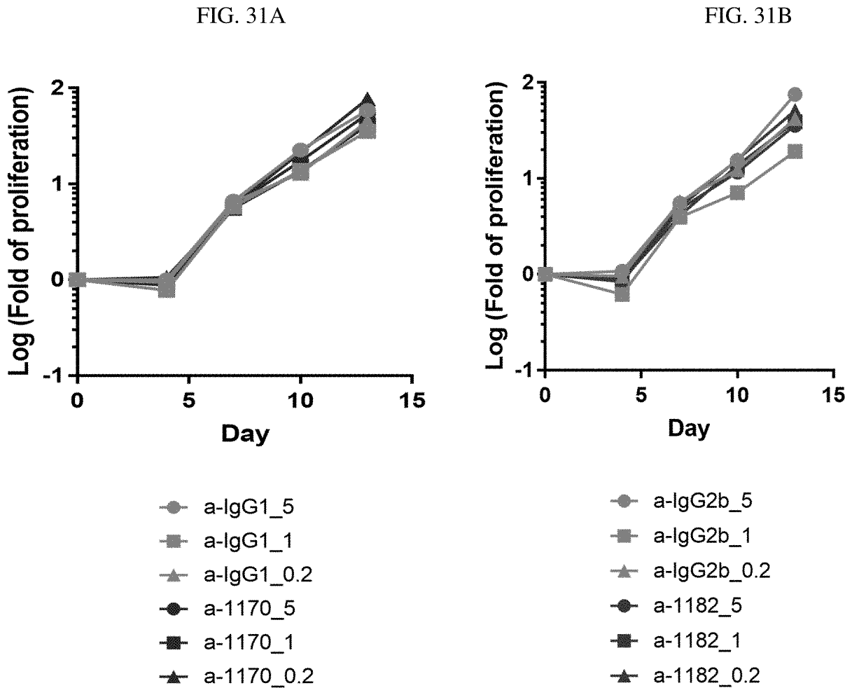

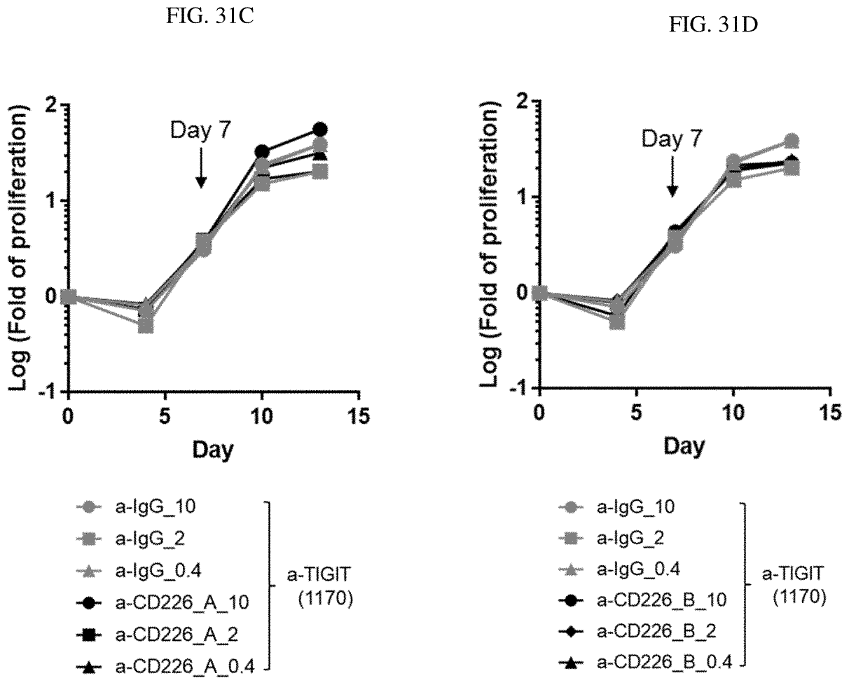

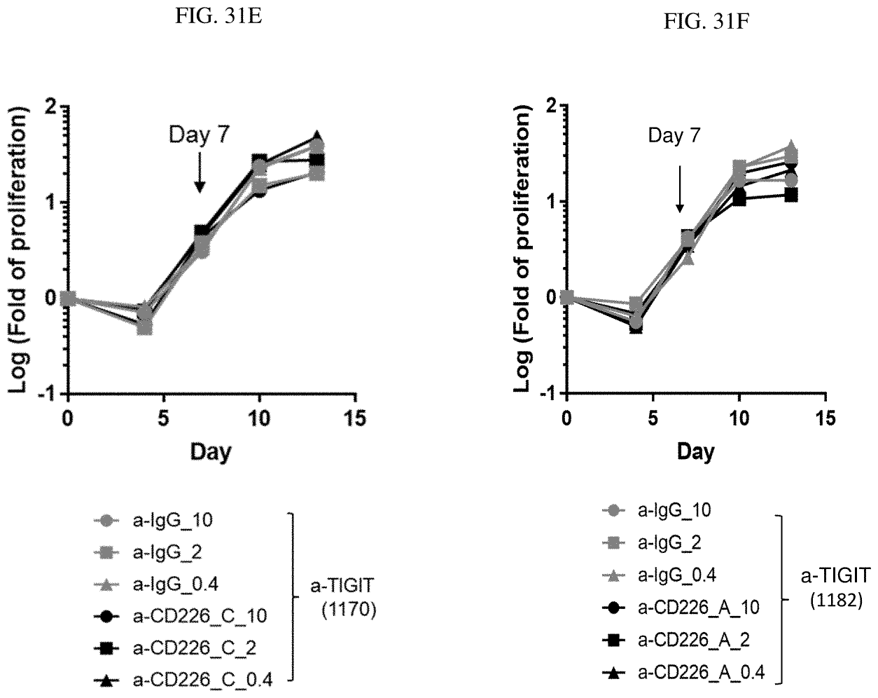

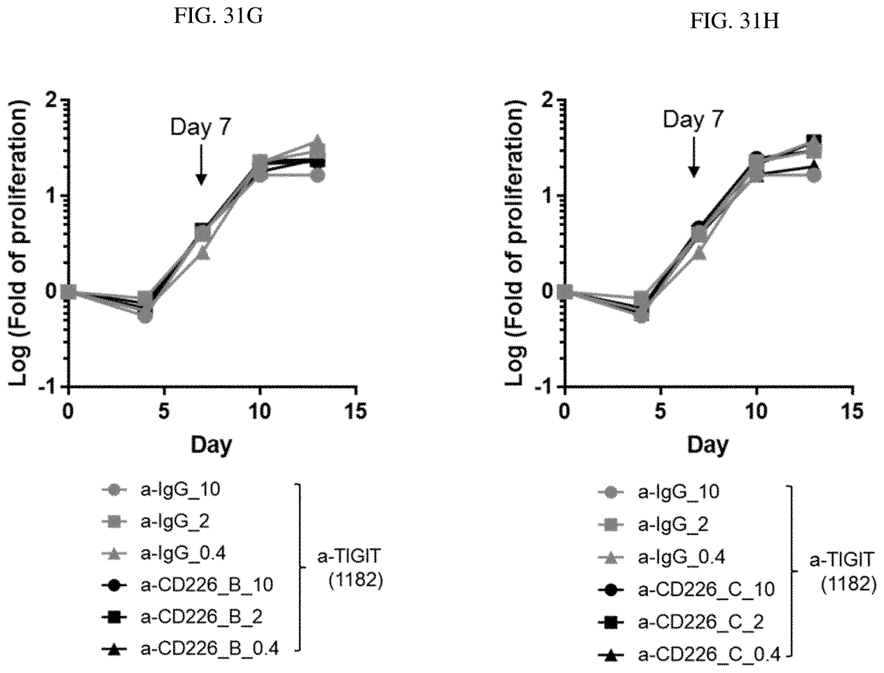

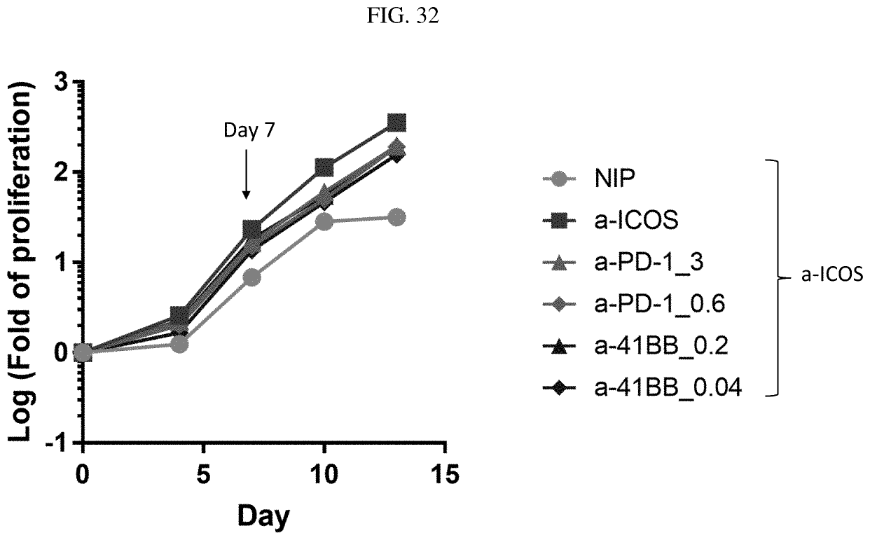

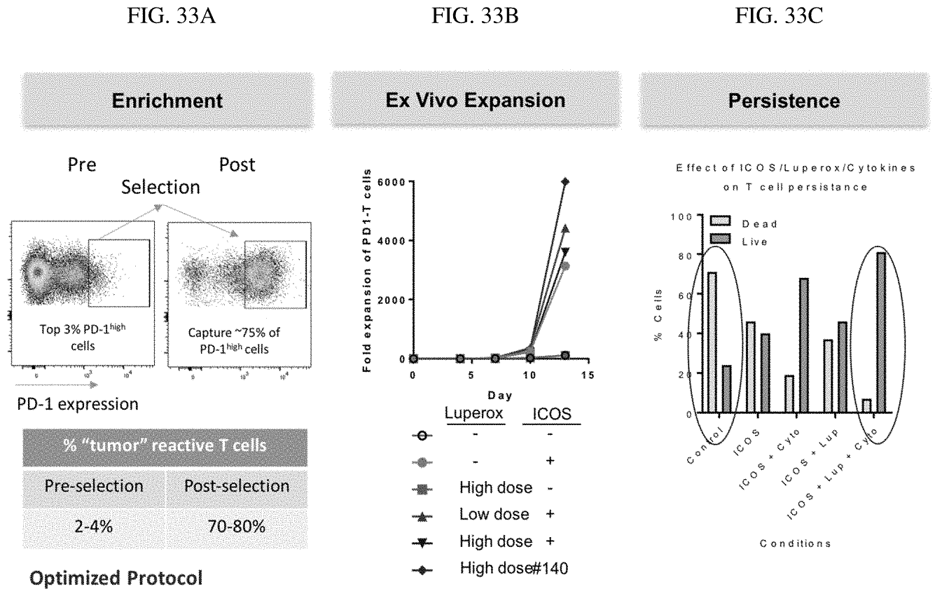

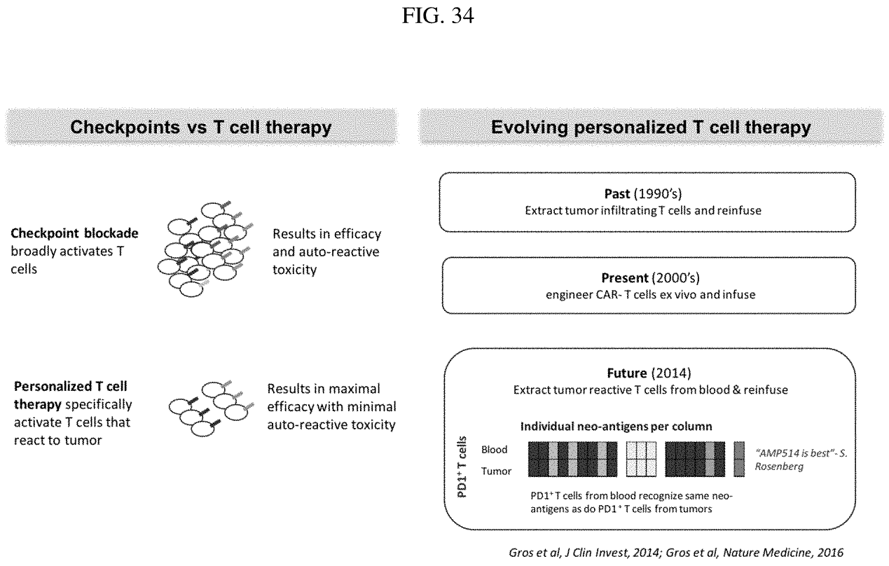

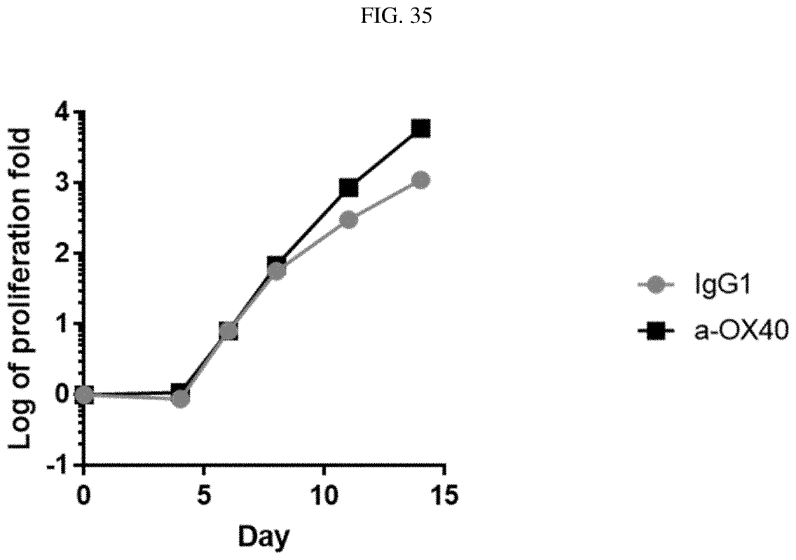

Analyses were performed to determine a range of antibodies that could be used in the successful expansion of PD-1 expressing T cells using the methods disclosed herein. In such analyses, the priming step was changed from that in Example 4, such that the antibodies to be tested replaced the α-ICOS in coating the non-treated 24-well plate. The antibodies to be tested were added at varying concentrations. The antibodies tested included: (a) α-CD137 (41BB), (b) α-PD-1, (c) α-LAG3, (d) α-TIM3 (62GL), (e) α-TIM3 (F9S), (f) OX40 (g) α-TIGIT (1170), and (h) α-TIGIT (1182). Experiments were also performed using the following combinations of priming antibodies: (h) α-ICOS with α-CD137 (41BB), (i) α-TIGIT (1170) with α-CD226-A, (j) α-TIGIT (1170) with α-CD226-B, (k) α-TIGIT (1170) with α-CD226-C, (1) α-TIGIT (1182) with α-CD226-A, (m) α-TIGIT (1182) with α-CD226-B, and (n) α-TIGIT (1182) with α-CD226-C. None of these experiments showed activity for the expansion of PD-1 T cells (see FIGS. 27 through 31), with the exception of α-ICOS with α-CD137 (41BB) that showed activity, but no improvement over using α-ICOS alone (see FIG. 32). These results demonstrated that B7 family molecule ICOS uniquely modified cellular fitness of PD-1⁺ T cells that induce rubust expansion and it's quite different from that of TNF receptor family molecules, CD137 and OX40.

### Example 13. Proliferation and Activation of Primary Human T Cells Using Antibody-Conjugated Beads

Experiments were completed to assess the feasibility of activation of primary human T cells without the utilization of plate-bound anti-CD3 and anti-ICOS antibodies. For proof of concept, M280 and M450 tosylactivated polystyrene Dynabeads® (Invitrogen) were incubated with a range of anti-human CD3 antibody (MedImmune), anti-ICOS antibody (MedImmune), or a 1:4 ratio of anti-CD3:anti-ICOS antibodies (DualBeads). Efficiency of antibody coating was assessed indirectly by evaluating the total protein in the coating solution before and after bead coating, and absorbance was evaluated. For T cell activation controls, 96-well, non-tissue culture coated plates were pre-coated overnight with anti-CD3 (MedImmune), anti-ICOS (MedImmune), or a combination of the two antibodies.

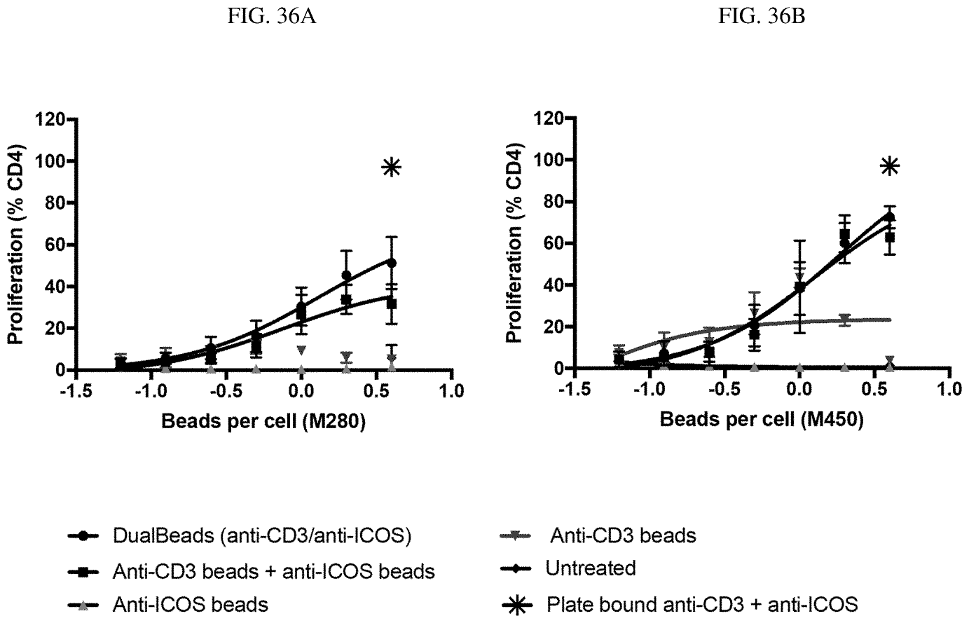

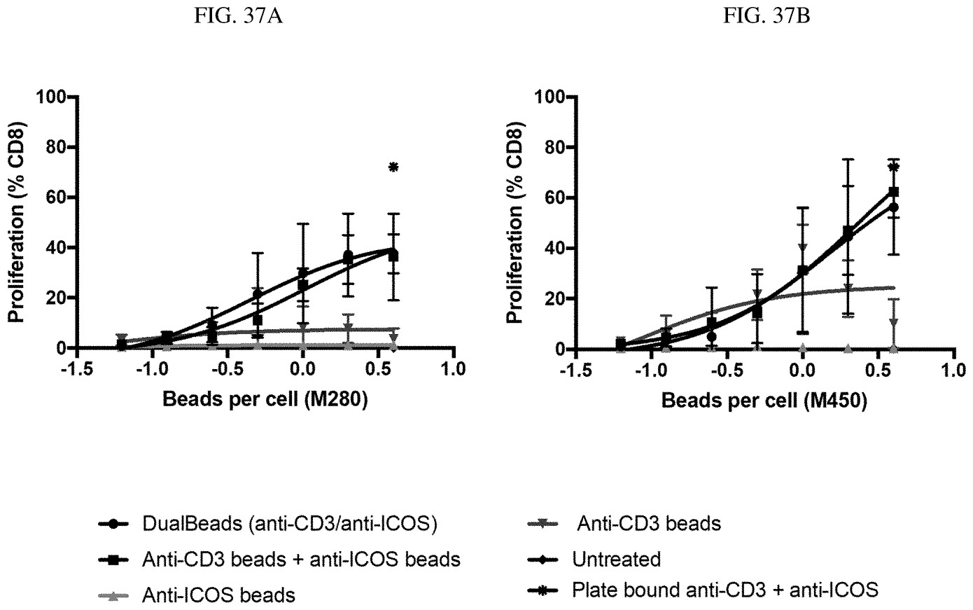

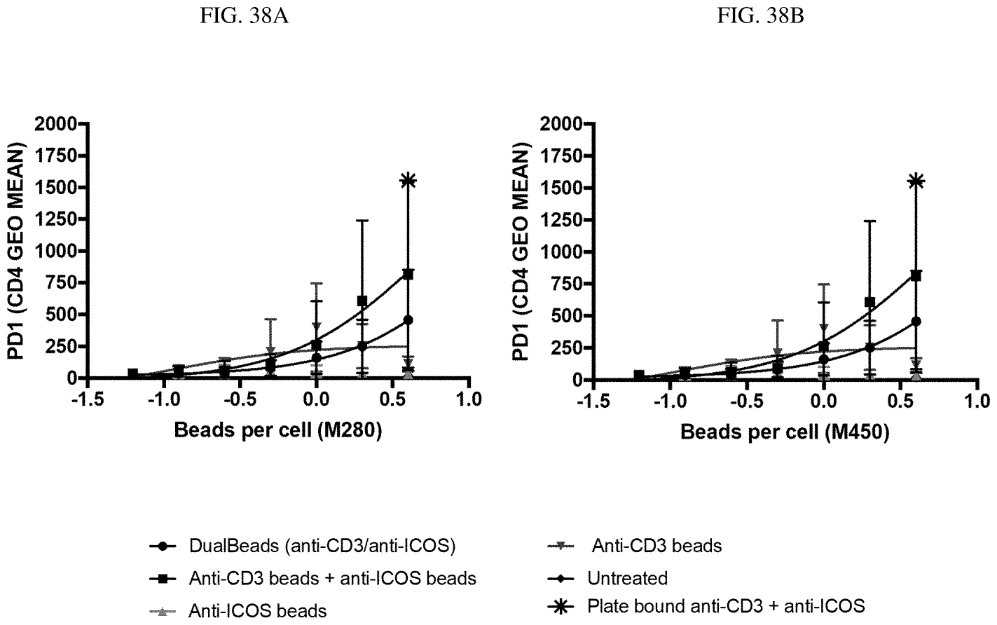

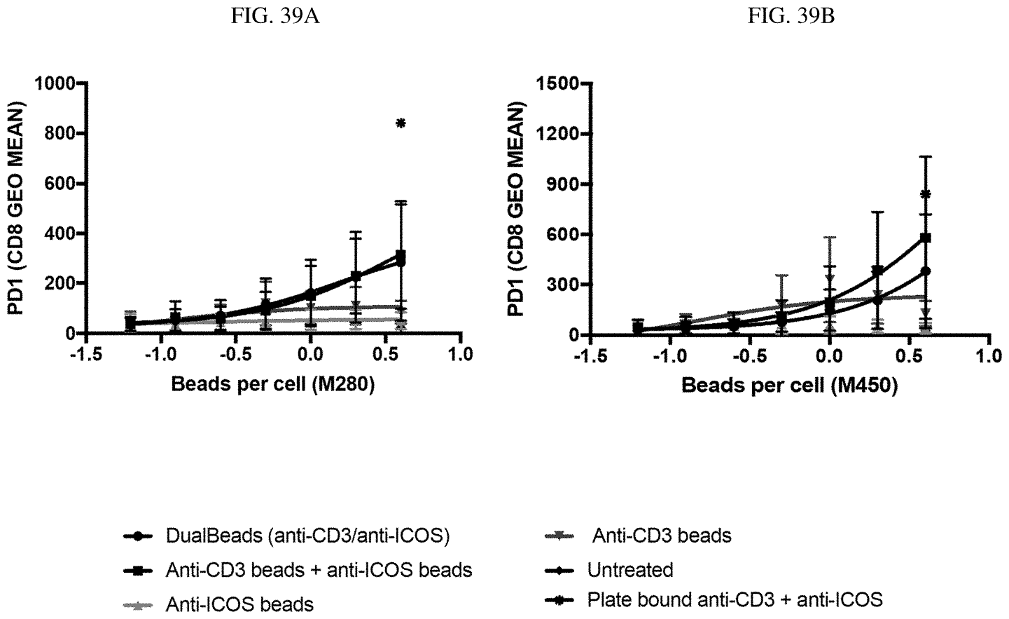

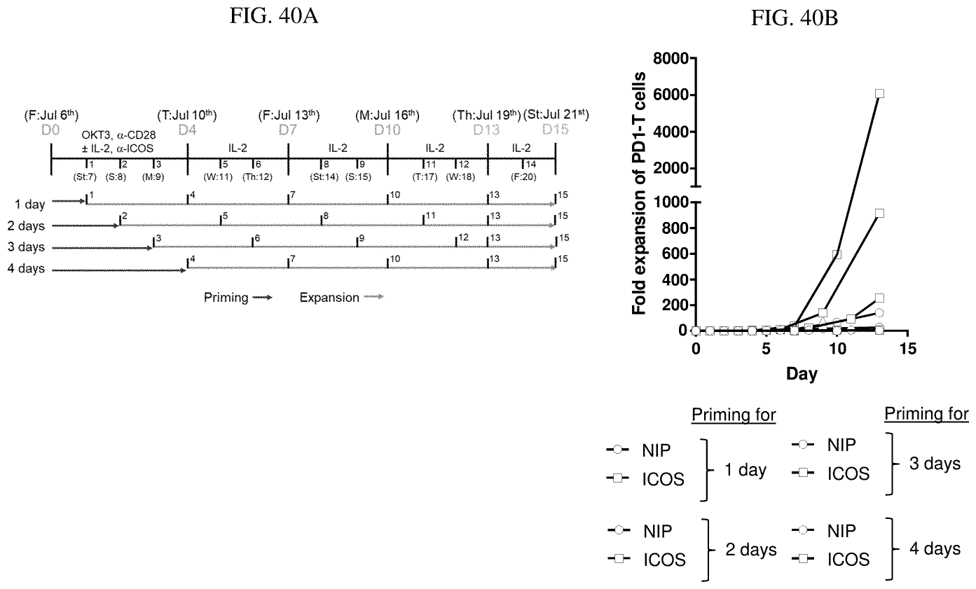

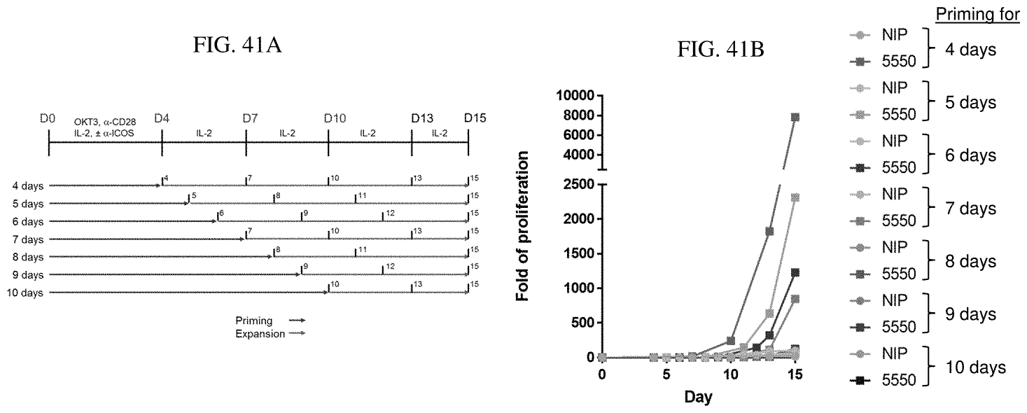

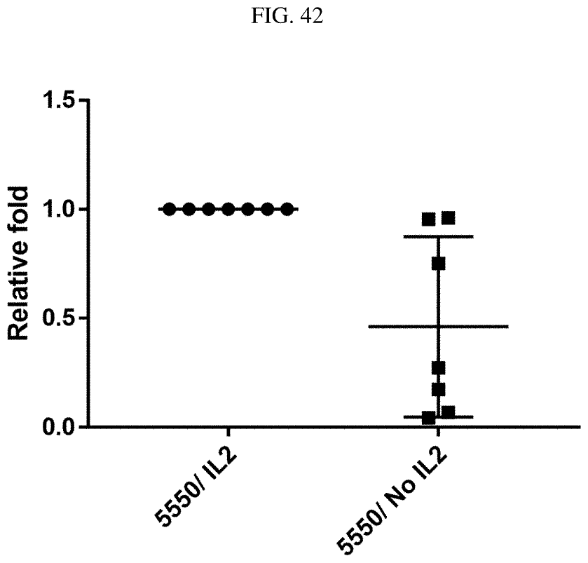

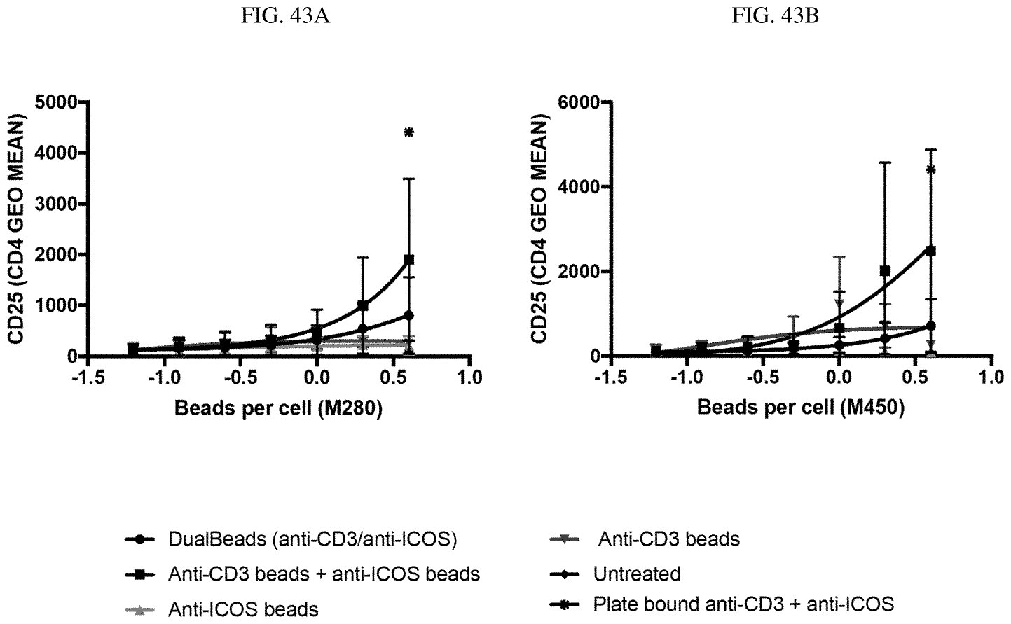

Whole blood was collected from four healthy human donors by informed consent, and total T cells were purified by negative selection (StemCell). Cells were labeled with carboxyfluorescein succinimidyl ester (CFSE) (Invitrogen) and 50,000 cells were seeded in media (RPMI 1640 with 10% fetal bovine serum with 1× penicillin/streptomycin) into 96-well tissue culture plates. Additionally, comparable numbers of CFSE-labeled cells were added to the plate-bound control plate described above. Serial dilutions of anti-CD3 beads, anti-ICOS beads, anti-CD3/anti-ICOS beads, or a combination of anti-CD3 beads and anti-ICOS beads were prepared at concentrations of 4e6, 2e6, 1e6, 0.5e6, 0.25e6, 0.125e6, or 0.062e6 beads per mL of media. 100 μL of beads were added to appropriate wells of cells for final bead:cell ratios of 4:1, 2:1, 1:1, 1:2, 1:4, 1:8, and 1:16. All plates with cells were incubated for approximately 90 hours 37° C. and 5% CO₂. After incubation, cells were stained for flow cytometry analysis. FIGS. 36-44 illustrate that beads coated with anti-ICOS and anti-CD3 molecules can induce proliferation and activate CD4 and CD8 cells. This activation of the T cells is, in some cases, comparable to plate-bound reagents.

### Example 14. Effects of ICOS on T Cell Expansion

Towards developing a scalable, rapid expansion protocol (REP) for PD-1⁺ T cells, various effects of inducible T-cell costimulator (ICOS) on T cell proliferation were investigated. The effect of ICOS agonism on mitochondrial mass and glucose consumption was tested. T cells enriched from leukapheresis products as described in Examples 1 and 2 were expanded as described in Example 4. On day 4, the seeded cells were harvested from the 24-well plate, counted and the effect of ICOS agonism on mitochondrial mass and glucose consumption was tested. In order to test Mitochondrial mass, 100,000 cells in expansion media (5% CST-SR XVivo15 with 500 U/ml IL-2) were seeded into 96-well U bottom tissue culture plates. The plates with cells were incubated for approximately 30 min at 37° C. and 5% CO₂ prior to add reagent. After incubation, Mitotracker was added into cell culture at final concentration 50 nM. The cells were incubated for 35 min at 37° C. After incubation, the cells were washed with PBS twice and then quickly stained with fluorescence-labeled anti-CD3, CD4 and CD8 antibodies in PBS for 15 min for flow cytometry analysis. The re-suspended cells in PBS were immediately run flow cytometry within 1 hour.

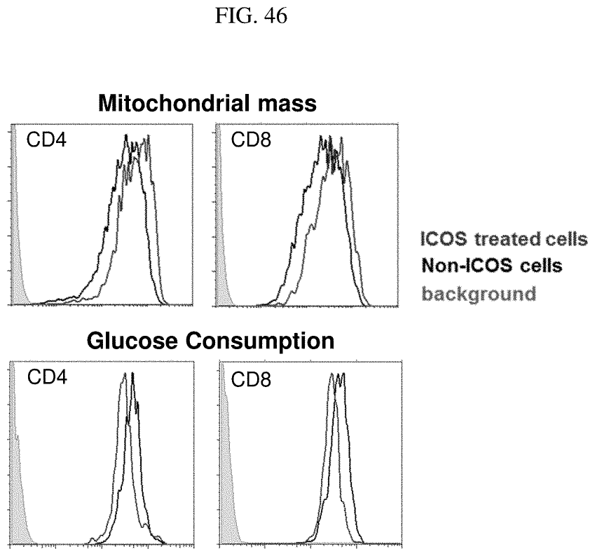

To test Glucose consumption, 100,000 cells in glucose-free media (Agilent Seahorse XF Assay Medium Modified DMEM, 0 mM glucose) were seeded into 96-well U bottom tissue culture plates. The plates with cells were incubated for 30 min at 37° C. and 5% CO₂ prior to add reagent. After incubation, 2-NBDG was added into cell culture at final concentration 50 ug/ml. The cells were incubated for 35 min at 37° C. After incubation, the cells were washed with PBS twice and then quickly stained with fluorescence-labeled anti-CD3, CD4 and CD8 antibodies in PBS for 15 min for flow cytometry analysis. The re-suspended cells in PBS were immediately run flow cytometry within 1 hour. As shown in FIG. 46, ICOS agonism increased mitochondrial mass in both CD4 and CD8 T cells. The mitcondrial mass increasement is associated with a more proliferative phenotype. ICOS agonism was also observed to reduce initial consumption of glucose, suggesting that ICOS treatment drives oxidative phosphorylation rather than glycolysis; oxidative phosphorylation is associated with enhanced T cell persistence.

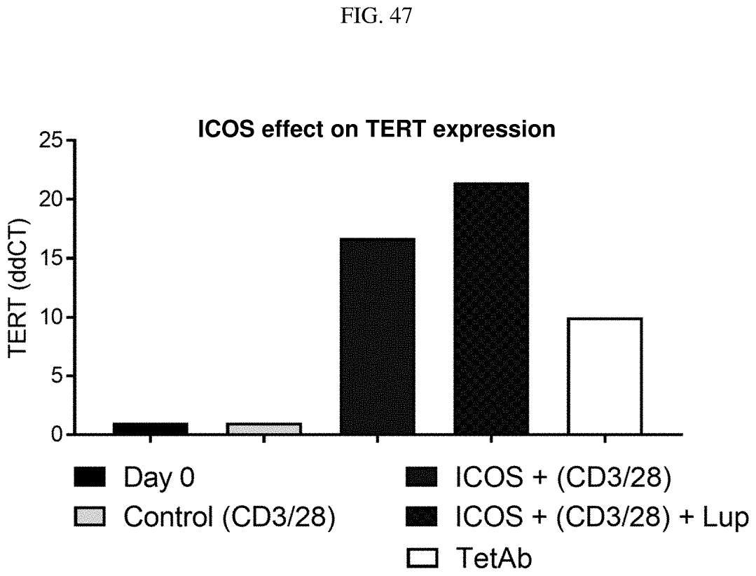

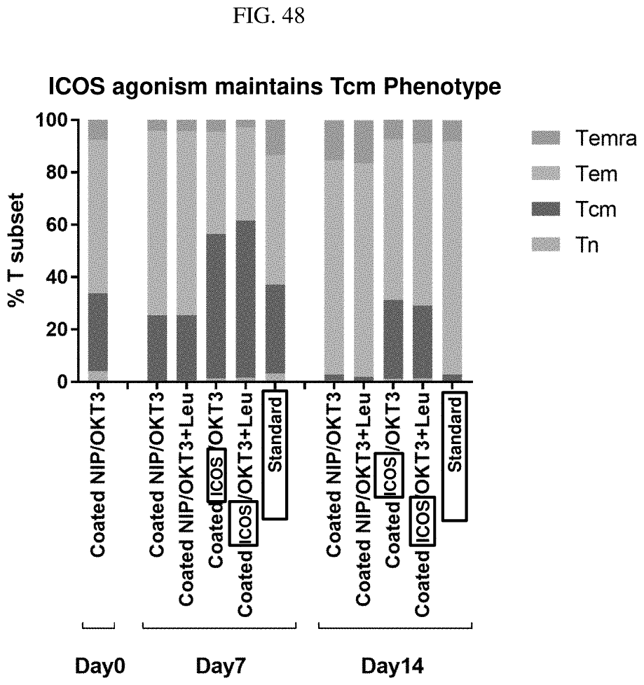

The effects ICOS, ICOS+Luperox, or TetAb on human telomerase reverse transcriptase (hTERT) expression during rapid expansion of enriched T cells were also assessed by quantitative reverse transcriptase polymerase chain reaction (qRT-PCR). RNA was extracted from cells collected on days 0 and 10 using RNeasy Plus Mini and QIAshredder Kits (P/N 74134 and 79656, Qiagen). First strand cDNA was synthesized, then reverse transcriptase reactions were performed with Superscript III RT (P/N 18080-044, Invitrogen) using a Tetrad2 Peltier Thermal Cycler (BioRad). Quantitative RT-PCR was performed using TaqMan® Gene Expression Assay for human TERT (Hs00972656_m1, Applied Biosystems) and endogenous control GAPDH (Hs02786624_g1, Applied Biosystems) with TaqMan® Fast Universal PCR Master Mix (P/N 4352042, Life Technologies) on a BioRad CFX96 Real-Time System (BioRad). The data was normalized to day 0, then delta delta CT was calculated and normalized to isotype control. Telomere elongation is associated with stem cell-like capacity of cells, and telomerase activity is associated with T cell renewal. As shown in FIG. 47, ICOS agonism increased TERT expression. Luperox was included based on its ability to increase mitochondrial mass and cell metabolism, which may help cells retain their proliferative and functional capacity. Indeed, the addition of Luperox to ICOS agonism increased TERT expression above ICOS agonism alone.

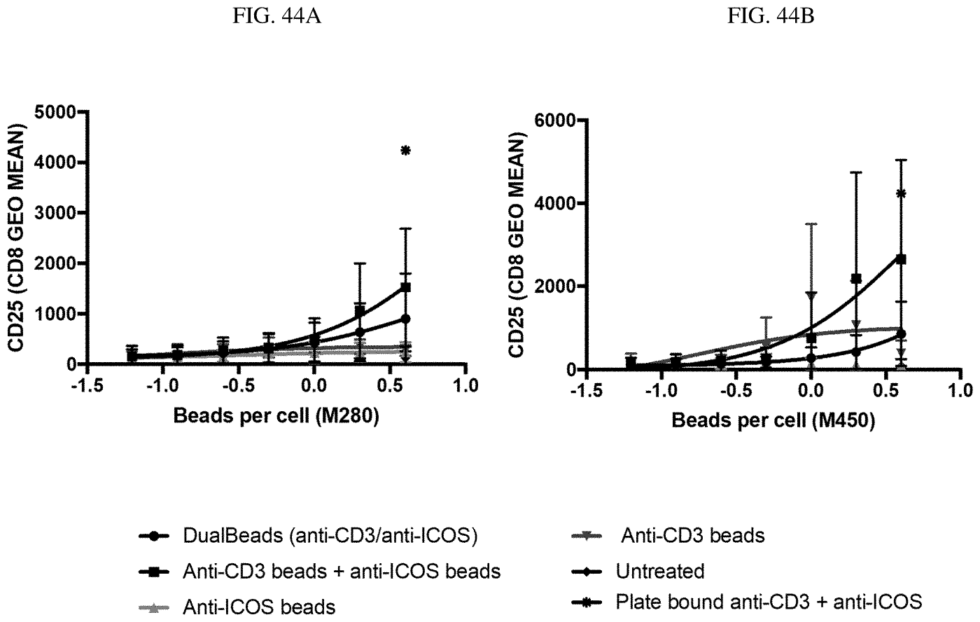

The effect of ICOS agonism on T-cell phenotype was also investigated. Initial CD8 T cells that survive T-cell contraction express an effector-memory cell (Tem) phenotype, whereas memory CD8 T cell populations found long after clearance of infection are predominantly composed of central-memory T cells (Tcm). Persistence of the transferred T cells is highly correlated with treatment outcome. Infusion of younger T cells such as T_{SCM} and T_{CM} phenotypes showed superior persistence and antitumor effects compared with T cells with the T_{EM} phenotype in both mice and humans. However, the ex vitro expansion of T cells using the standard REP is inevitably accompanied with differentiation toward T_{EM} cells. Therefore, most of the T cell grafts currently used in adoptive T cell therapy trials comprise T cells with excessive differentiation. In this study, T cells enriched from leukapheresis were expanded as described in Examples 4 and also with a standard rapid expansion protocol as described in Dudley et al., 2003, J. Immunother. 26(4): 332-42. On day 0, 7 and 14 of expansion, the cells were stained with fluorescence-conjugated anti-CD3, CD4, CD8, PD-1, CD45RA, and CCR7 to test T cell phenotypes. As shown in FIG. 44, ICOS agonism was shown to maintain the Tcm phenotype.

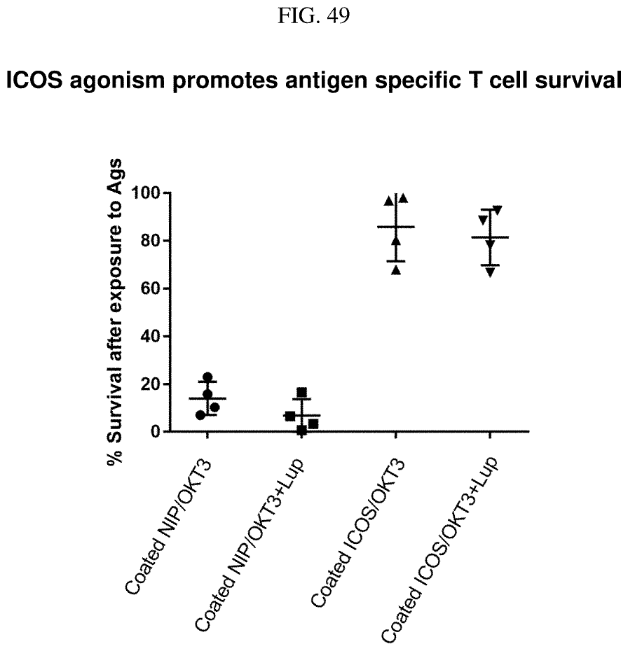

The effect of ICOS agonism on T cell survival in response to antigens was also tested. Expanded T cells in the absence of proper costimulation mediates cell death during restimulation with antigens. Resistance to antigen-induced cell death is one of key explanation of persistence and antitumor effects of T cells both in vitro and in vivo. PD-1+ T cells enriched from leukapheresis products as described in Examples 1 and 2 were expanded as described in Example 4. On day 14 of expansion, PD-1 T cells were co-cultured with T2 cell line loaded with or without CMVpp65(495-503) peptide for 5 hours and then tested the frequency of viable CMVpp65(495-503) peptide-specific T cells which were detected by fluorescent labelled CMVpp65(495-503) peptide-HLA-A*0201 dextramer. The T cells primed with ICOS but not without ICOS maintained original frequency of CMVpp65(495-503) peptide-specific T cells after stimulation with the antigen, suggesting ICOS agonism promotes antigen-specific T cell survival (FIG. 49).

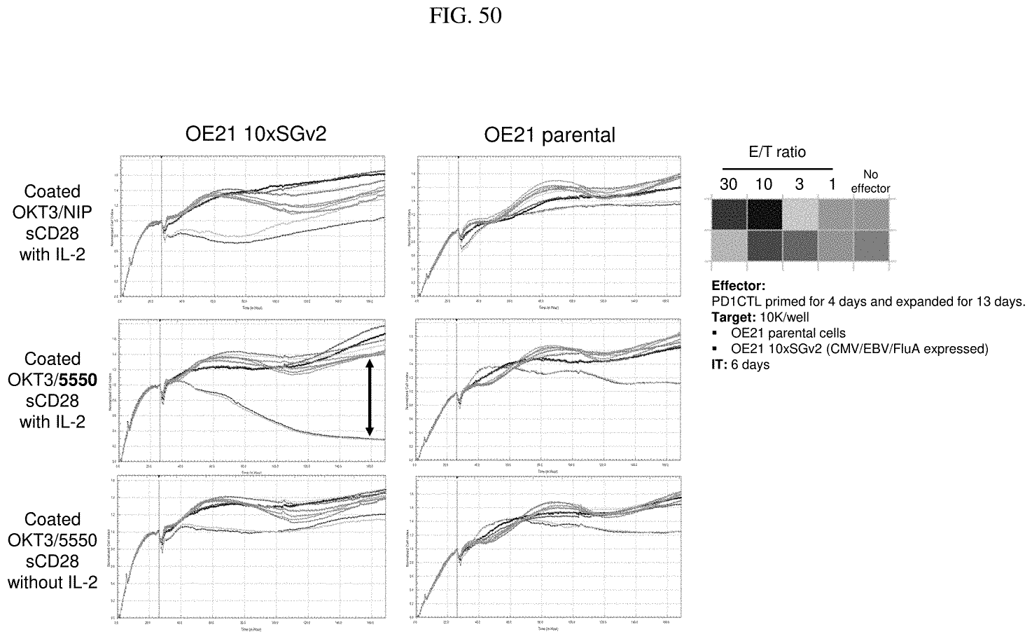

The effect of ICOS agonism on cytotoxic activity was tested. PD-1+ T cells enriched from leukapheresis products with HLA-A*0201+ and CMV+as described in Examples 1 and 2 were expanded as described in Example 4. On day 13, the cytotoxic activity of expanded T cells were tested by the xCELLigence Real-Time Cell Analysis (RTCA) assay. As shown in FIG. 50, ICOS agonism promotes cytotoxic activity of PD-1 T cells.

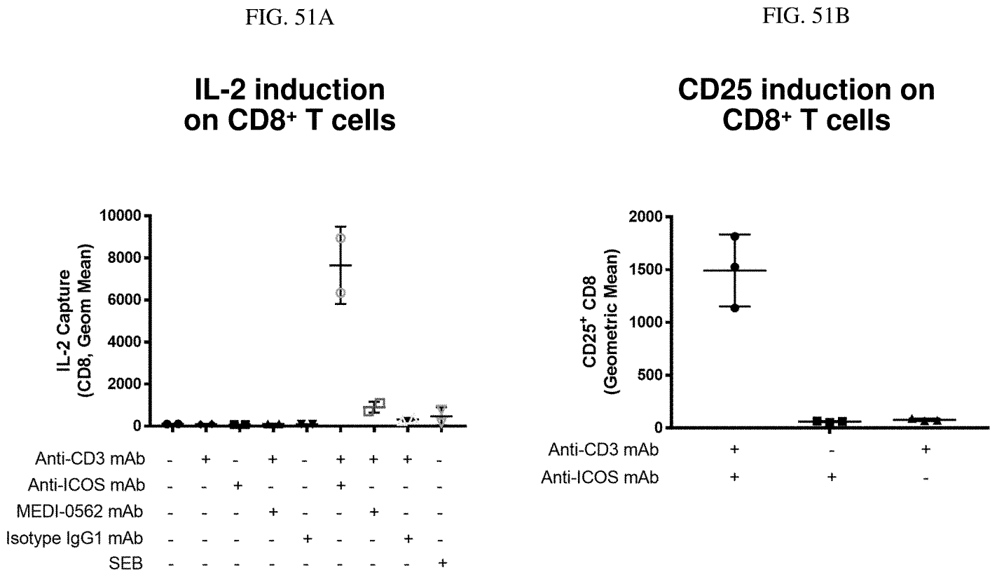

Comparison of the effects of an ICOS agonist and other T cell agonists during T cell activation/proliferation revealed that ICOS agonism uniquely drives IL-2 expression. Briefly, plates were coated overnight in PBS with 0.25 μg/mL anti-CD3 (clone OKT3) or 1 μg/mL of anti-ICOS, anti-OX40 (MEDI-0562) or IgG1 isotype control antibody. Total human primary T cells were isolated by negative selection from peripheral blood. 100,000 purified T cells were then added into each well of the pre-coated plates and shaken gently for four to seven hours at 37° C., 5% CO₂. IL-2 capture was assessed using an IL-2 secretion assay (Miltenyi Biotec). As a control for IL-2 production, 1 μg/mL Staphylococcal enterotoxin B (SEB) was added to separate wells. FIG. 51A shows CD8+IL-2 induction after treatment with 0.25 μg/mL monoclonal antibodies anti-CD3 (aCD3), anti-ICOS, anti-OX40 (MEDI0562), anti-ICOS+ anti-CD3, or MEDI0562+ anti-CD3), IgG1, or Staphylococcal enterotoxin B (SEB). In similar experiments, there was little to no detectable IL-2 in the supernatants from the anti-CD3+ anti-ICOS costimulation, suggesting that IL-2 is secreted and almost immediately captured by the concomitantly induced expression of IL-2 receptor alpha (CD25)(FIG. 51B).

### Example 15. Tumor Infiltrating Lymphocytes (TILs) from Patients with Multiple Type of Cancers Express PD-1

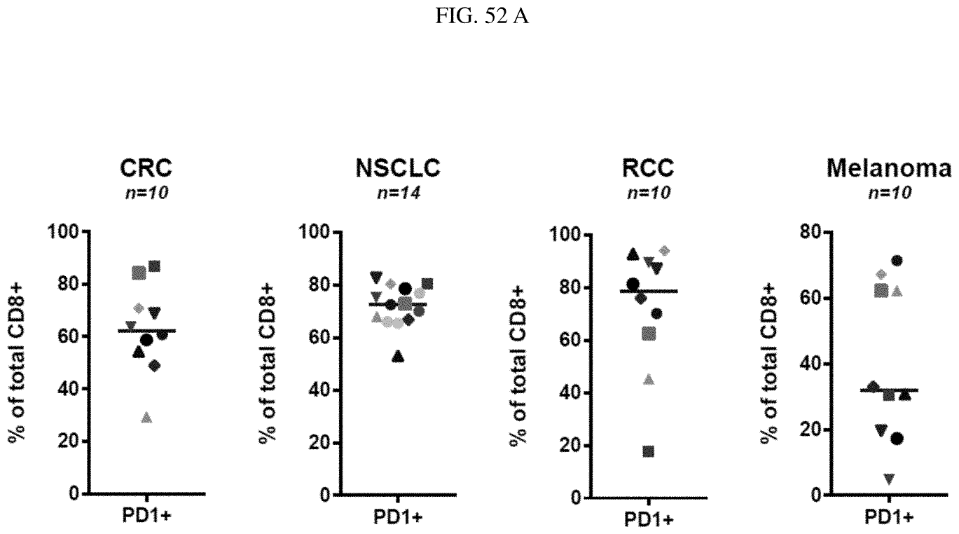

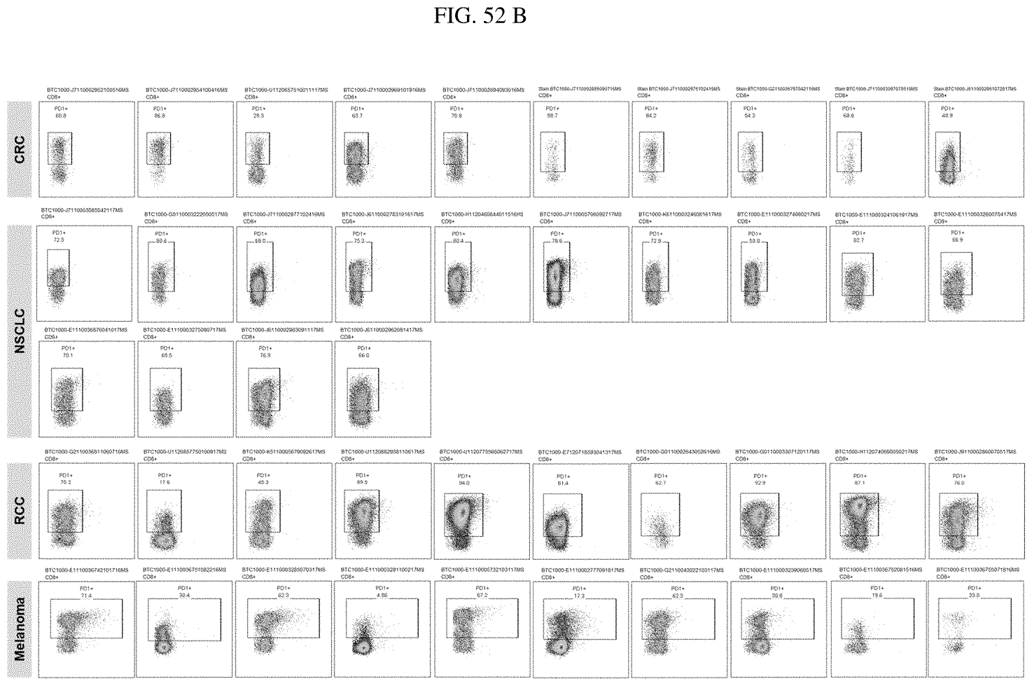

PD-1 expression in CD3+CD8+ T cells in the dissociated tumor tissues from multiple type of cancers was tested by flow cytometry. As shown FIGS. 52A and 52B, the majority of tumor infiltrating lymphocytes (TILs) express high level of PD-1. It has been reported that tumor reactive T cells are highly enriched in PD-1 expressing TILs, suggesting sources of PD-1+ T cells are not limited to PBMC in peripheral blood, but also can be selected from other tissues including TILs.

### Example 16. Induction of PD-1 Expressing T Cells in Peripheral Blood by Anti-CTLA-4 Antibody and by the Combination of Anti-PD-L1+Anti-CTLA-4 Antibodies

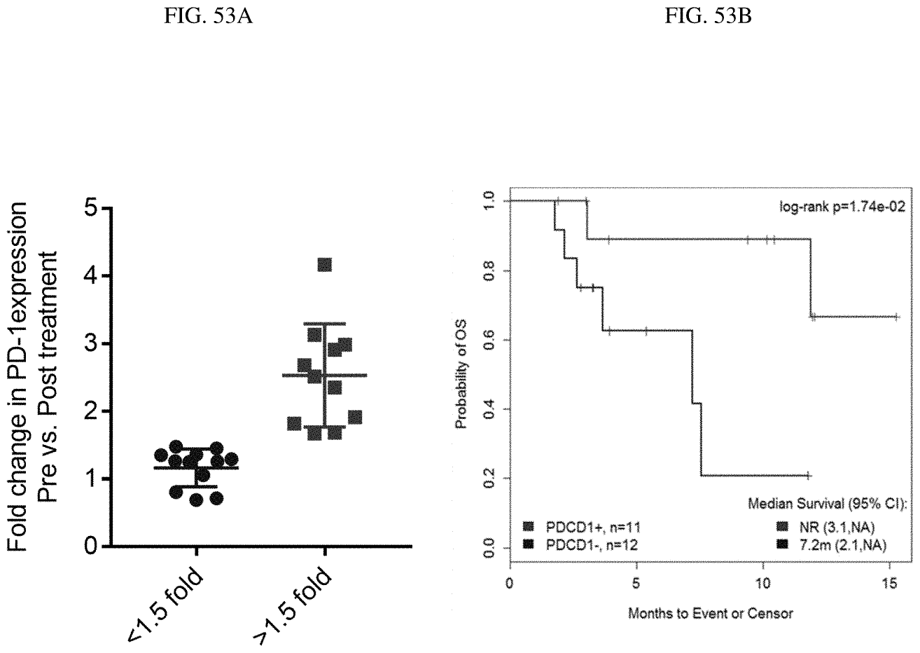

The effect of anti-CTLA-4 antibody (tremelimumab) pre-treatment on PD-1 expression in peripheral blood was tested by Taqman qRT-PCR. As shown in FIG. 53A, tremelimumab pre-treatment induced a >1.5-fold increase PD-1 expression in peripheral blood in a group of patients (squares) after 9-10 days, which correlated with improved overall survival (OS) in a clinical trial; NTC02527434 (FIG. 53B). Based on these results, tremelimumab can be used to enhance mobilization of PD-1⁺ T cells into the peripheral blood towards the treatment of cancer.

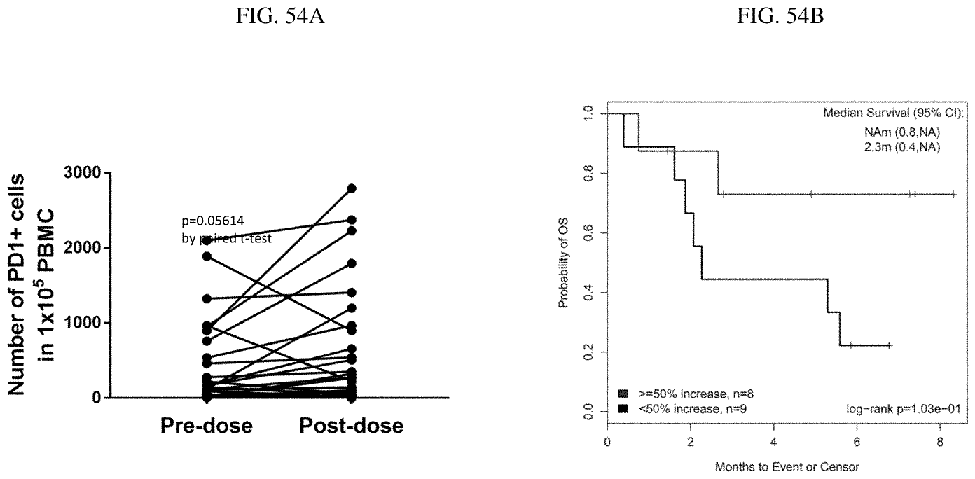

The effect of durvalumab and tremelimumab pretreatment on the number of PD-1 expressing T cells in peripheral blood was also tested by Flow cytometry. As shown in FIG. 54A, durvamumab/tremelimumab pre-treatment increased the number of PD-1⁺ T cells in patient peripheral blood on day 8 and the >=50% increased chohort showed improved OS compared to <50% increased cohort (FIG. 54B).

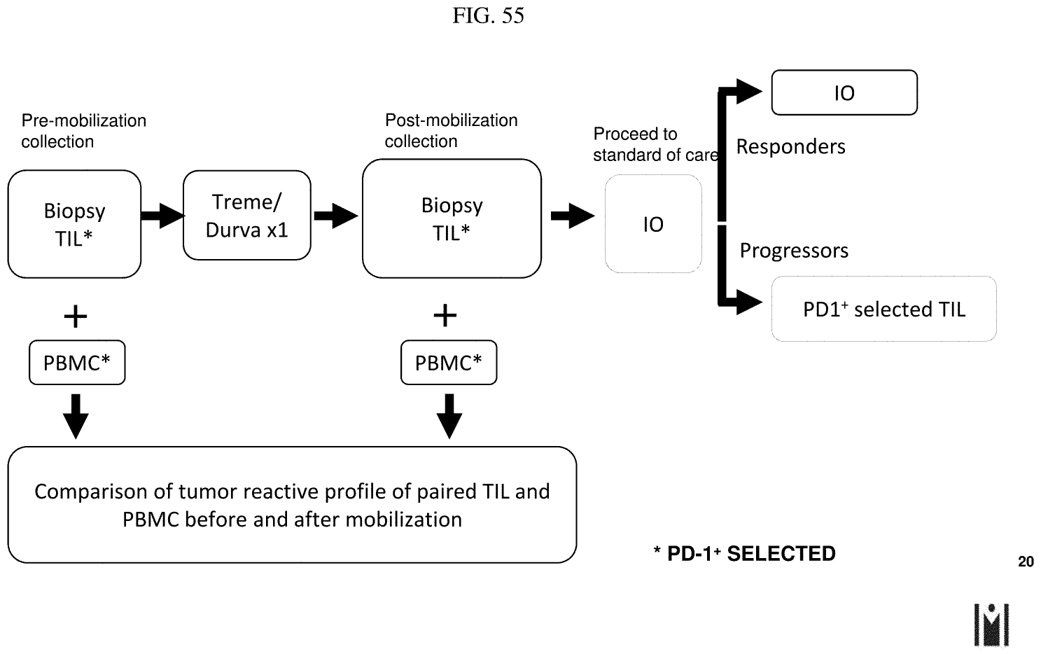

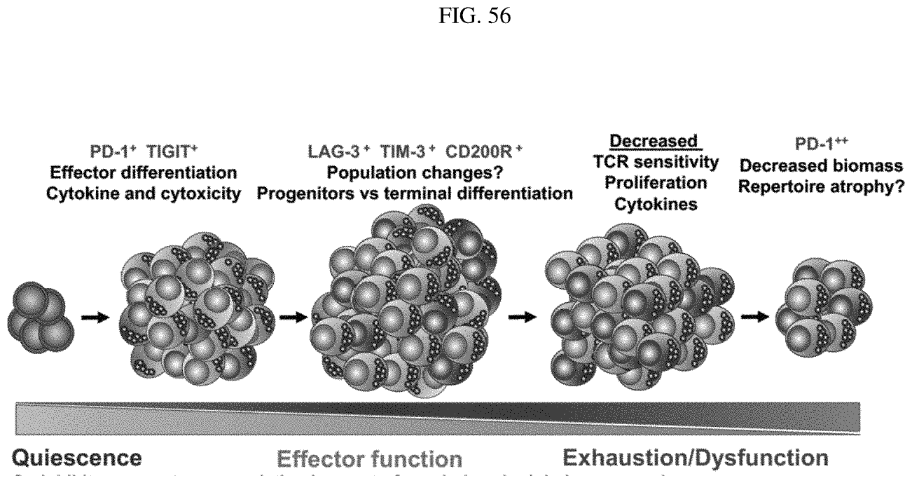

An exemplary clinical trial protocol for treatment of cancer using durvamumab/tremelimumab mobilized T cells is shown in FIG. 55. The primary aim of this study is to evaluate the mobilization and capture method of tumor reactive T cells from periphery. Patients are biopsied pre-mobilization, and peripheral blood mononuclear cells (PBMC) collected for later analysis. Patients are then mobilized using durvamumab/tremelimumab and biopsied tumor and peripheral blood mononuclear cells (PBMC) are collected post-mobilization and then frozen.

Following harvesting of TIL, patients are treated with an immunotherapy. Patients responsive to this treatment continue. Patients with disease progression after immunotherapy are treated with pre- and/or post-mobilization cells as follows. The NeoAg reactivity profiles of PD-1+ cells enriched from PBMC and TILs collected pre-mobilization and post-mobilization are compared to determine treatment protocol. In particular, if NeoAg reactivity overlaps, the patient is treated with PBMC first, reserving TIL as backup. If no overlap in reactivity, TIL are used for treatment.

While the disclosure has been described in terms of various embodiments, it is understood that variations and modifications will occur to those skilled in the art. Therefore, it is intended that the appended claims cover all such equivalent variations that come within the scope of the disclosure as claimed. In addition, the section headings used herein are for organizational purposes only and are not to be construed as limiting the subject matter described.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

What is claimed is:

1. A method of isolating T cells expressing programmed cell death 1 (PD-1) from a cell population, comprising:
(a) contacting the cell population with an amount of an anti-PD-1 antibody to produce an antibody-cell mixture, wherein the anti-PD-1 antibody comprises a capture moiety, and wherein the capture moiety is connected to the anti-PD-1 antibody via a linker;
(b) contacting the antibody-cell mixture with an amount of magnetic beads, wherein the magnetic beads are capable of specifically binding the capture moiety on the anti-PD-1 antibody to produce a bead mixture;
(c) passing the bead mixture through a magnetic field to isolate the magnetic beads and PD-1 expressing T cells bound thereto from the bead mixture; and
(d) eluting the PD-1 expressing T cells from the magnetic field to isolate T cells expressing PD-1.

2. The method of claim 1 further comprising selecting a PD-1 expression level of the T cells expressing PD-1 isolated in step (d) by adjusting one or more of:
(i) the concentration of the anti-PD-1 antibody in the antibody-cell mixture;
(ii) the length of the linker;
(iii) the stoichiometric ratio of capture moiety to anti-PD-1 antibody (CAR);
(iv) the ratio of anti-PD-1 antibody comprising a capture moiety to unmodified anti-PD-1 antibody;
(v) the temperature at which either step (a) and/or step (b) is carried out;
(vi) the antibody-cell mixture and/or in the bead mixture;
(vii) the concentration of magnetic beads in the bead mixture;
(viii) the flow rate at which the bead mixture is passed through the magnetic field;
(ix) the length of time between the production of the antibody-cell mixture and step (b);
(x) the length of time between production of the bead mixture and step (c); or
(xi) the magnetic field strength.

3. The method of claim 1, wherein the T cells are CD8+ T cells.

4. The method of claim 1, wherein the T cells are CD4+ T cells.

5. The method of claim 1, wherein the capture moiety is biotin.

6. The method of claim 1, wherein the linker is between about 1 Å and about 50 Å in length.

7. The method of claim 2, wherein increasing the length of the linker decreases the PD-1 expression level of the T cells expressing PD-1 isolated in step (d).

8. The method of claim 2 wherein increasing the length of the linker increases the yield of T cells expressing PD-1 isolated in step (d).

9. The method of claim 2, wherein the CAR is between 1 and 8.

10. The method of claim 2, wherein increasing the CAR decreases the PD-1 expression level of the T cells expressing PD-1 isolated in step (d).

11. The method of claim 2, wherein increasing CAR increases the yield of T cells expressing PD-1 isolated in step (d).

12. The method of claim 2, wherein the concentration of T cells in the antibody-cell mixture or bead mixture is between 20 million cells per mL and 500 million cells per mL.

13. The method of claim 1, wherein the cell population is obtained from a healthy subject.

14. The method of claim 1, wherein the cell population is obtained from a subject with cancer.

15. The method of claim 1, wherein the concentration of the magnetic beads in the bead mixture is between 1 μL per 1×10⁷ cells and 30 μL per 1×10⁷ cells.

16. The method of claim 2, wherein increasing the concentration of the magnetic beads in the bead mixture decreases the PD-1 expression level of the T cells expressing PD-1 isolated in step (d).

17. The method of claim 2, wherein increasing the concentration of the magnetic beads in the bead mixture increases the yield of T cells expressing PD-1 isolated in step (d).

18. The method of claim 1, wherein the anti-PD-1 antibody is LO115 or MEDI0680.

19. The method of claim 1, wherein the concentration of the anti-PD-1 antibody in the antibody-cell mixture is between 0.1 μg/mL and 10 μg/mL.

20. The method of claim 19, wherein the concentration of the anti-PD-1 antibody in the antibody-cell mixture is between 0.5 μg/mL and 5 μg/mL.

21. The method of claim 18, wherein:
(a) the anti-PD-1 antibody is LO115, and the concentration of the anti-PD-1 antibody in the antibody-cell mixture is between 0.01 μg/mL and 1 μg/mL; or
(b) the anti-PD-1 antibody is MEDI0680, and the concentration of the anti-PD-1 antibody in the antibody-cell mixture is between 0.5 μg/mL to 5 μg/mL.

22. The method of claim 2, wherein increasing the concentration of the anti-PD-1 antibody in the antibody-cell mixture decreases the PD-1 expression level of the T cells expressing PD-1 isolated in step (d).

23. The method of claim 2, wherein increasing the concentration of the anti-PD-1 antibody in the antibody-cell mixture increases the yield of T cells expressing PD-1 isolated in step (d).

24. The method of claim 1, wherein the steps of passing the bead mixture through a magnetic field and eluting the PD-1 expressing T cells from the magnetic field comprise:
(a) passing the bead mixture through the magnetic field at a high flow rate and/or low magnetic field intensity;
(b) eluting PD-1 expressing T cells from the magnetic field to isolate T cells having a high PD-1 expression level;
(c) passing a primary negative fraction of the bead mixture remaining after elution of the T cells having a high PD-1 expression level through the magnetic field at an intermediate flow rate and/or intermediate magnetic field intensity;
(d) eluting PD-1 expressing T cells from the magnetic field to isolate T cells having an intermediate PD-1 expression level;
(e) passing a secondary negative fraction of the bead mixture remaining after elution of the T cells having an intermediate PD-1 expression level through the magnetic field at a low flow rate and/or high magnetic field intensity; and
(f) eluting PD-1 expressing T cells from the magnetic field to isolate T cells having a low PD-1 expression level.

25. The method of claim 1, wherein the steps of passing the bead mixture through a magnetic field and eluting the PD-1 expressing T cells from the magnetic field comprise:
(a) passing the bead mixture through the magnetic field at a low flow rate and/or high magnetic field intensity to produce a first captured bead mixture fraction and a discarded bead mixture fraction;
(b) eluting the first captured bead mixture fraction from the magnetic field;
(c) passing the first captured bead mixture fraction through the magnetic field at an intermediate flow rate and/or intermediate magnetic field intensity to produce a secondary captured bead mixture fraction and a first negative bead mixture fraction, wherein the first negative bead mixture fraction comprises T cells having a low PD-1 expression level;
(d) passing the secondary captured bead mixture fraction through the magnetic field at a high flow rate and/or low magnetic filed intensity to produce a tertiary captured bead mixture fraction and a second negative bead mixture fraction, wherein the tertiary captured bead mixture fraction comprises T cells having a high PD-1 expression level and the second negative bead mixture fraction comprises T cells having an intermediate PD-1 expression level.

26. The method of claim 2, wherein the PD-1 expression level of the isolated T cells expressing PD-1 is adjusted according to Formula I:
$$N ( label ) = \frac{N * α * {[ Ab ]}_{i}}{{K}_{d} + {[ Ab ]}_{i}} ,$$
wherein:
N(label) is the number of expected label molecules on the T cells expressing PD-1;
N is the number of PD-1 antigen-binding sites on the T cells expressing PD-1;
[Ab]ᵢ is the total concentration of the anti-PD-1 antibody in the antibody-cell mixture;
α is the ratio of anti-PD-1 antibody with accessible capture moiety to the total anti-PD-1 antibody; and
K_{d} is the dissociation constant of the anti-PD-1 antibody at the incubation temperature of step (a).

27. The method of claim 24, wherein a is adjusted by changing one or more of:
(i) the length of the linker;
(ii) the stoichiometric ratio of capture moiety to anti-PD-1 antibody (CAR);
(iii) the ratio of anti-PD-1 antibody comprising a capture moiety to unmodified anti-PD-1 antibody.

28. A method for ex vivo T cell expansion, comprising:
(a) priming a sample of T cells expressing PD-1 isolated according to the method of claim 1, wherein the step of priming comprises:
(i) coating a culture plate on day −1 with priming factors;
(ii) seeding the population of T cells expressing PD-1 on day 0, wherein the step of seeding comprises:
(1) adding a base media to the coated culture plate; and/or
(2) adding an amount of isolated and/or enriched T cells to the base media to produce a seeding mixture in the coated culture plate;
(b) harvesting the primed T cells expressing PD-1;
(c) placing the harvested T cells expressing PD-1 in a seeding mixture and placing the seeding mixture into a non-treated culture plate;
(d) culturing the T cells expressing PD-1 in the seeding mixture;
(e) harvesting the T cells expressing PD-1 from the cultured seeding mixture;
(f) repeating steps (b)-(e) until a target number of expanded T cells is obtained.

29. The method of claim 28, wherein the priming factors include OKT3, soluble α-CD28, α-ICOS, α-ICOS (#140), α-LAGS, α-CD137, α-OX40, or any combination thereof.

30. The method of claim 28, further comprising adding additives to the base media, wherein the additives comprise IL-2, α-TIGIT, Iso, α-CD226, α-CD28, α-TIM3, α-LAG3, α-PD-1, α-OX40, Luperox, Bezafibrate, or any combination thereof.

31. A method of treating a subject comprising administering to the subject a therapeutically effective amount of T cells expressing PD-1 isolated according to the method of claim 1.

32. A method for ex vivo T cell expansion, comprising:
(a) contacting a population of T cells with beads conjugated with anti-CD3 antibody, anti-ICOS antibody, or a combination thereof;
(b) incubating the bead and T cell mixture to expand the T cell population.

33. The method of claim 32, wherein the T cell population comprises activated CD4 and CD8 cells.

34. A method of treating a subject comprising administering to the subject a therapeutically effective amount of T cells expressing PD-1;
wherein the T cells expressing PD-1 are isolated by:
(a) contacting the cell population with an amount of an anti-PD-1 antibody to produce an antibody-cell mixture, wherein the anti-PD-1 antibody comprises a capture moiety, and wherein the capture moiety is connected to the anti-PD-1 antibody via a linker;
(b) contacting the antibody-cell mixture with an amount of magnetic beads, wherein the magnetic beads are capable of specifically binding the capture moiety on the anti-PD-1 antibody to produce a bead mixture;
(c) passing the bead mixture through a magnetic field to isolate the magnetic beads and PD-1 expressing T cells bound thereto from the bead mixture; and
(d) eluting the PD-1 expressing T cells from the magnetic field to isolate T cells expressing PD-1;
wherein a PD-1 expression level of the T cells expressing PD-1 isolated in step (d) is selected by adjusting one or more of:
(i) the concentration of the anti-PD-1 antibody in the antibody-cell mixture;
(ii) the length of the linker;
(iii) the stoichiometric ratio of capture moiety to anti-PD-1 antibody (CAR);
(iv) the ratio of anti-PD-1 antibody comprising a capture moiety to unmodified anti-PD-1 antibody;
(v) the temperature at which either step (a) and/or step (b) is carried out;
(vi) the concentration of T cells in the cell population;
(vii) the concentration of magnetic beads in the bead mixture;
(viii) the flow rate at which the bead mixture is passed through the magnetic field;
(ix) the length of time between the production of the antibody-cell mixture and step (b);
(x) the length of time between production of the bead mixture and step (c); or
(xi) the force applied by the magnetic field; and
wherein the population of T cells expressing PD-1 is subjected to ex vivo expansion by:
(a) priming a sample of T cells expressing PD-1 isolated according to the method of claim 1, wherein the step of priming comprises:
(i) coating a culture plate on day −1 with priming factors;
(ii) seeding the population of T cells expressing PD-1 on day 0, wherein the step of seeding comprises:
(1) adding a base media to the coated culture plate; and/or
(2) adding an amount of isolated and/or enriched T cells to the base media to produce a seeding mixture in the coated culture plate;
(b) harvesting the primed T cells expressing PD-1;
(c) placing the harvested T cells expressing PD-1 in a seeding mixture and placing the seeding mixture into a non-treated culture plate;
(d) culturing the T cells expressing PD-1 in the seeding mixture;
(e) harvesting the T cells expressing PD-1 from the cultured seeding mixture;
(f) repeating steps (b)-(e) until a target number of expanded T cells is obtained.

35. The method of any one claim 28,
wherein the priming factors include an ICOS agonist.

36. The method of claim 35, wherein the ICOS agonist is an anti-ICOS antibody.

37. The method of claim 35, wherein T cells are primed for 4 days.

38. The method of claim 28, wherein culturing of T cells after priming is conducted in the presence of IL-2.

39. A method of treating cancer in a subject, the method comprising:
(a) administering an anti-CTLA-4 antibody to the subject in an amount effective to mobilize PD-1⁺ tumor-infiltrating lymphocytes (TIL) into the peripheral blood;
(b) harvesting PD-1⁺ TIL from the peripheral blood of the subject;
(c) expanding the harvested PD-1⁺ TIL; and
(d) administering the expanded PD-1⁺ TIL to the subject.

40. The method of claim 39, wherein the harvested PD-1⁺ TIL are expanded according to the method of claim 28.

41. The method of claim 39, wherein the anti-CTLA-4 antibody is tremelimumab.
